(19) United States
(12) Patent Application Publication
(10) Pub. No.: US 2021/0346039 A1
(43) Pub. Date: Nov. 11, 2021

(54) INTERNAL CAROTID ARTERY THROMBECTOMY DEVICES AND METHODS

(71) Applicant: MARBLEHEAD MEDICAL LLC, Rochester, MN (US)

(72) Inventors: David Kallmes, Rochester, MN (US); Waleed Brinjikji, Rochester, MN (US); Brady Hatcher, Rogers, MN (US); Randy Beyreis, Rogers, MN (US)

(21) Appl. No.: 17/283,467

(22) PCT Filed: Oct. 9, 2019
(86) PCT No.: PCT/US2019/055451

§ 371 Date: Apr. 7, 2021

Related U.S. Application Data
(60) Provisional application No. 62/744,104, filed on Oct. 10, 2018.
(60) Provisional application No. 62/781,738, filed on Dec. 19, 2018.
(60) Provisional application No. 62/816,766, filed on Mar. 11, 2019.

(51) Int. Cl. A61B 17/22

(52) U.S. Cl. CPC A61B 17/22; A61B 2017/3486; A61B 2017/22067

(57) ABSTRACT

The disclosure includes a balloon guiding sheath that includes an elongated sheath having a proximal end, a distal end opposite the proximal end, an inner tube extending between the proximal end and the distal end, an outer tube surrounding the inner tube and extending between the proximal end and the distal end, an access port located adjacent in the proximal end, a distal port located adjacent the distal end, and a working lumen extending through the elongated sheath between the access port and the distal port. The balloon guiding sheath also includes an inflatable balloon located on an outer surface of the elongated sheath adjacent the distal end, the inflatable balloon being fluidly coupled to an inflation lumen extending between the inflatable balloon and an inflation port located adjacent the proximal end.

### CROSS REFERENCE TO RELATED APPLICATION

The entire contents of the following application are incorporated by reference herein: U.S. Patent Application No. 62/332,495; having a filing date of May 6, 2016; and entitled INTERNAL CAROTID ARTERY THROMBECTOMY DEVICES AND METHODS.

The entire contents of the following application are incorporated by reference herein: International Patent Application PCT/US2017/031311; having an international filing date of May 5, 2017 and a priority date of May 6, 2016; and entitled INTERNAL CAROTID ARTERY THROMBECTOMY DEVICES AND METHODS.

The entire contents of the following application are incorporated by reference herein: U.S. Patent Application No. 62/700,173; having a filing date of Jul. 18, 2018; and entitled INTERNAL CAROTID ARTERY THROMBECTOMY DEVICES AND METHODS.

The entire contents of the following application are incorporated by reference herein: PCT Patent Application No. PCT/US19/42181; having an international filing date of Jul. 17, 2019 and a priority date of Jul. 18, 2018; and entitled INTERNAL CAROTID ARTERY THROMBECTOMY DEVICES AND METHODS.

The entire contents of the following application are incorporated by reference herein: U.S. Patent Application No. 62/744,104; having a filing date of Oct. 10, 2018; and entitled INTERNAL CAROTID ARTERY THROMBECTOMY DEVICES AND METHODS.

The entire contents of the following application are incorporated by reference herein: U.S. Patent Application No. 62/781,738; having a filing date of Dec. 19, 2018; and entitled INTERNAL CAROTID ARTERY THROMBECTOMY DEVICES AND METHODS.

The entire contents of the following application are incorporated by reference herein: U.S. patent application Ser. No. 16/319,764; having a filing date of Jan. 22, 2019; and entitled INTERNAL CAROTID ARTERY THROMBECTOMY DEVICES AND METHODS.

The entire contents of the following application are incorporated by reference herein: U.S. Patent Application No. 62/816,766; having a filing date of Mar. 11, 2019; and entitled INTERNAL CAROTID ARTERY THROMBECTOMY DEVICES AND METHODS.

### BACKGROUND

### Field of Invention

The invention relates generally to medical devices and methods of use. Embodiments of the invention include devices for performing thrombectomy or embolectomy in the internal carotid artery and other vessels of a patient.

### Description of Related Art

Acute Ischemic Stroke (AIS) can be caused by thrombus, embolus or other occlusions in regions of the internal carotid artery (ICA) such as the Petrous part, Cavernous part or Cerebral part. Approaches for performing thrombectomy or embolectomy to treat AIS include positioning a balloon guiding catheter in the carotid artery at a location upstream from the occlusion, typically at a proximal location in the artery such as the cervical part. After the balloon is inflated to provide antegrade blood flow cessation, suction can be applied to the catheter to retrieve the embolus. Thrombectomy tools such as stent retrievers can also be delivered directly to the embolus through the guiding catheter to break up the embolus and enhance the retrieval process.

These thrombectomy procedures may involve placing a sheath through an arteriotomy in the patient's common femoral artery, and delivering the guiding catheter to the ICA through the sheath. For example, an 8-9 French (Fr) inner diameter (ID) (0.015-0.118 inches) sheath having a length on the order of twenty-five centimeters can be used to provide the access to the arterial tree through the arteriotomy. A balloon guiding catheter having a 7-8 Fr outer diameter (OD) (0.092-0.105 inches), commonly about ninety centimeters in length, can then be delivered to the ICA through the sheath. A 10-11 Fr (0.131-0.144 inch) arteriotomy may be required for the sheath during procedures of these types. Unfortunately these relatively large arteriotomies can enhance the risk of bleeding, especially since patient's undergoing these procedures may be receiving thrombolytics that may increase the risks of hemorrhagic complications.

Relatively small diameter distal access aspiration catheters (e.g., up to about 0.087 inch OD) are sometimes used during thrombectomy in the ICA. Such distal aspiration catheters include the ACE 68 from Penumbra, Inc. and the Sophia Plus from Microvention, Inc. For example, during these procedures the distal aspiration catheter can inserted with the end positioned at the distal middle cerebral artery. Other thrombecotomy tools such as stent retrievers are sometimes delivered to the intracranial vasculature through distal access catheters used in this manner. However, balloon guiding catheters have IDs that are too small to accommodate these distal aspiration catheters. Other known balloon guide catheters include the MO.MA Ultra and Cello devices from Medtronic, Inc., and the Flowgate2 device from Stryker Neurovascular. The relatively long period of time required to place a sheath and then a balloon guide catheter can detract from the benefits of this treatment.

Stents and other endovascular tools are sometimes placed in the ICA or other vasculature using guiding sheaths that do not have balloons. Guiding sheaths are typically about ninety centimeters in length. These devices act as a combination of access sheath and guiding catheter. The need for a separate sheath is obviated by the use of these guiding sheaths since they are sufficiently long to provide access to the target vessel. Although guiding sheaths do not provide arterial occlusion, they can be rapidly placed.

There is a continuing need for improved devices and methods for performing mechanical revascularization such as thrombectomy and embolectomy in the ICA and other vasculature. In particular, there is a need for such devices and methods that provide enhanced efficacy. Devices and methods of these types that can improve the efficiency of health care delivery would be especially desirable.

### SUMMARY

The present disclosure describes a balloon guiding sheath that includes an elongated sheath having a proximal end, a distal end, a proximal portion defining a first outer diameter, and a distal portion defining a second outer diameter. The balloon guiding sheath may include an access port located on the proximal end, a distal port located on the distal end, a working lumen extending through the elongated sheath between the access port and the distal port, an inflation port on the proximal end, an inflatable balloon coupled to the distal end, and an inflation lumen extending in the elongated sheath between the inflation port and the balloon. The elongated sheath may be sized and configured to enable direct insertion into a patient's vasculature through an arteriotomy in the patient's femoral artery to position the balloon at a target site.

The first outer diameter may be greater than the second outer diameter. In some embodiments, the first outer diameter is approximately equal to 0.123 inches. However, in some embodiments, the first outer diameter is approximately equal to 0.137 inches. Additionally, in some embodiments, the second outer diameter is approximately equal to 0.104 inches such that the elongated sheath fits through an 8 Fr opening. Even still, in some embodiments, the second outer diameter is approximately equal to 0.124 inches such that the elongated sheath fits through a 10 Fr opening.

The distal portion of the elongated sheath may include an inner tube and an outer tube that surrounds the inner tube. Furthermore, the distal portion of the elongated sheath may include a reinforcement layer located between the inner tube and the outer tube. The reinforcement layer arranged and configured to enable flow of at least one of fluid and media through the inflation lumen.

The inflation lumen may include a proximal inflation lumen extending from the inflation port to a middle portion located between the proximal portion and the distal portion. The inflation lumen may also include a distal inflation lumen extending from the middle portion to the balloon, whereby the distal inflation lumen is located between the inner tube and the outer tube.

In some embodiments, the proximal portion of the working lumen defines a first inner diameter approximately equal to 0.090 inches. In some embodiments, the first inner diameter is greater than or equal to 0.101 inches and less than or equal to 0.113 inches. In some embodiments, the first inner diameter is approximately equal to 0.113 inches.

Additionally, the distal portion of the working lumen may define a second inner diameter greater than or equal to 0.087 inches and less than or equal to 0.090 inches. In some embodiments, the second inner diameter greater than or equal to 0.101 inches and less than or equal to 0.113 inches. In some embodiments, the second inner diameter is approximately equal to 0.101 inches.

In some embodiments, the working lumen defines an inner diameter extending from the proximal end to the distal end. The inner diameter may be approximately equal to 0.088 inches. The proximal portion of the working lumen may equal approximately 85 centimeters in length. The distal portion of the working lumen may equal approximately 10 centimeters in length. The elongated sheath may define a working length approximately equal to 95 centimeters.

Furthermore, in some embodiments, the working length is long enough to enable the distal end to reach at least a cervical portion of a patient's internal carotid artery from the femoral artery. Even still, in some embodiments, the working length is long enough to enable the distal end to reach at least a petrous portion of a patient's internal carotid artery from the femoral artery. As well, in some embodiments, the working length is long enough to enable the distal end to reach at least a cavernous portion of a patient's internal carotid artery from the femoral artery.

In some embodiments, the balloon extends around and beyond a distal tip of the elongated sheath and defines a funnel-shaped opening into the distal port when the balloon is in an inflated state such that the balloon does not occlude the working lumen of the distal port. The elongated sheath may be arranged and configured to have sufficient stiffness and tip flexibility to enable insertion of the working length of the sheath into a patient's vasculature through an arteriotomy in the patient's carotid artery to position the distal port at a target site in at least one of a petrous portion of a patient's internal carotid artery, a cavernous portion of a patient's internal carotid artery, and a cerebral portion of a patient's internal carotid artery.

The disclosure also includes a balloon guiding sheath that comprises an elongated sheath having a proximal end, a distal end, a proximal portion defining a first outer diameter, and a distal portion defining a second outer diameter. The balloon guiding sheath may also include an access port located on the proximal end, a distal port located on the distal end, a working lumen extending through the elongated sheath between the access port and the distal port, an inflation port on the proximal end, an inflatable balloon coupled to the distal end, and an inflation lumen extending through the elongated sheath between the inflation port and the balloon. The inflation lumen may comprise a distal inflation port extending through an endwall of the elongated sheath, wherein the inflation lumen is not in fluid communication with the working lumen between the access port and the distal port. The elongated sheath may be sized and configured to enable direct insertion into a patient's vasculature through an arteriotomy in the patient's femoral artery to position the balloon at a target site. In some embodiments, the first outer diameter is greater than the second outer diameter.

In some embodiments, the balloon guiding sheath comprises a first inflation hole extending from the inflation lumen through a sidewall of the elongated sheath, wherein when a guide wire is inserted into the inflation lumen and out through the distal inflation port, the distal inflation port thereby creates a seal against the guide wire. Additionally, the inflation lumen may be arranged and configured to enable flow of at least one of fluid and media through the inflation lumen into the first inflation hole and into the balloon to thereby inflate the balloon.

In some embodiments, the inflation lumen is a first inflation lumen. The balloon guiding sheath may further comprise a second inflation lumen extending through the elongated sheath between the inflation port and the balloon. The second inflation lumen may comprise a second distal inflation port extending through the endwall of the elongated sheath, wherein the second inflation lumen is not in fluid communication with the working lumen between the access port and the distal port. The balloon guiding sheath may also include a second inflation hole extending from the second inflation lumen through the sidewall of the elongated sheath, wherein when a second guide wire is inserted into the second inflation lumen and out through the second distal inflation port, the second distal inflation port thereby creates a seal against the second guide wire. The second inflation lumen may be arranged and configured to enable flow of at least one of fluid and media through the second inflation lumen into the second inflation hole and into the balloon to thereby inflate the balloon.

The elongated sheath may define a central axis extending from the proximal end to the distal end, wherein at least a portion of the working lumen overlaps the central axis of the elongated sheath, and wherein the inflation lumen does not overlap the central axis of the elongated sheath. In some embodiments, the elongated sheath does not have a generally constant outer diameter along its working length.

The elongated sheath may define a working length long enough to enable the distal end to reach at least a cervical portion of a patient's internal carotid artery from the carotid artery. In some embodiments, the working length is long enough to enable the distal end to reach a cavernous portion of the patient's internal carotid artery from the femoral artery. Even still, in some embodiments, the elongated sheath is arranged and configured to have sufficient stiffness and tip flexibility to enable insertion of the working length of the sheath into a patient's vasculature through an arteriotomy in the patient's carotid artery to position the distal port at a target site in at least one of a petrous portion of a patient's internal carotid artery, a cavernous portion of the patient's internal carotid artery, and a cerebral portion of the patient's internal carotid artery.

The disclosure also includes a method for using a balloon guiding sheath comprising an elongated sheath having a proximal end, a distal end, a proximal portion defining a first outer diameter, and a distal portion defining a second outer diameter that is less than the first outer diameter, the guiding sheath including an access port located on the proximal end, a distal port located on the distal end, a working lumen extending through the elongated sheath between the access port and the distal port, an inflation port on the proximal end, an inflatable balloon coupled to the distal end, and an inflation lumen extending in the elongated sheath between the inflation port and the balloon. The method may include inserting the guiding sheath directly into a patient's vasculature through an arteriotomy in a patient's femoral artery, advancing the guiding sheath through the patient's vasculature and positioning the distal end in a target site of a patient's internal carotid artery, and inflating the balloon via the inflation lumen. The target site may be a cervical portion of the internal carotid artery, a petrous portion of the internal carotid artery, and/or a cavernous portion of the internal carotid artery.

Methods may include applying relatively low pressure to the access port to suction an embolus, deflating the balloon, and withdrawing the guiding sheath through the arteriotomy in the carotid artery. Methods may also include inserting a tool into the guiding sheath through the access port after positioning the distal end at the target site, advancing the tool through the guiding sheath, actuating the tool to retrieve an embolus, withdrawing the tool from the guiding sheath, deflating the balloon, and withdrawing the guiding sheath through the arteriotomy in the carotid artery.

The distal portion of the elongated sheath may comprise an inner tube and an outer tube that surrounds the inner tube. The inflation lumen may comprise a proximal inflation lumen extending from the inflation port to a middle portion located between the proximal portion and the distal portion, and a distal inflation lumen extending from the middle portion to the balloon. The distal inflation lumen may be located between the inner tube and the outer tube.

In some embodiments, inflating the balloon via the inflation lumen occurs in response to: inserting a guide wire into the working lumen, and sealing an outer surface of the guide wire against an inner surface of the working lumen adjacent the distal port. The elongated sheath further may comprise at least one inflation hole extending from the working lumen through a sidewall of the elongated sheath. In such embodiments, the method may include flowing at least one of fluid and media through a space within the inflation lumen between the guide wire and an inner surface of the inflation lumen and into the at least one inflation hole and into the balloon to thereby inflate the balloon.

Methods may also include removing the guide wire from the working lumen. In response to removing the guide wire from the working lumen, methods may include deflating the balloon.

The disclosure may also include a balloon guiding sheath that includes an elongated sheath having a proximal end, a distal end, a proximal portion defining a first outer diameter, and a distal portion defining a second outer diameter; an access port located on the proximal end; a distal port located on the distal end; a working lumen extending through the elongated sheath between the access port and the distal port; an inflation port on the proximal end; an inflatable balloon coupled to the distal end; and an inflation lumen extending in the elongated sheath between the inflation port and the balloon. The elongated sheath may be sized and configured to enable direct insertion into a patient's vasculature through an arteriotomy in the patient's femoral artery to position the balloon at a target site.

The first outer diameter may be greater than the second outer diameter. In some embodiments, the distal portion of the elongated sheath comprises an inner tube and an outer tube that surrounds the inner tube. The distal portion of the elongated sheath may include a reinforcement layer located between the inner tube and the outer tube, the reinforcement layer arranged and configured to enable flow of at least one of fluid and media through the inflation lumen.

The inflation lumen may comprise a proximal inflation lumen extending from the inflation port to a middle portion located between the proximal portion and the distal portion. The inflation lumen may also include a distal inflation lumen extending from the middle portion to the balloon, the distal inflation lumen located between the inner tube and the outer tube.

In some embodiments, the first outer diameter is approximately equal to 0.123 inches. Yet, in some embodiments, the second outer diameter is approximately equal to 0.104 inches such that the elongated sheath fits through an 8 Fr opening. Even still, in some embodiments, the proximal portion of the working lumen defines a first inner diameter approximately equal to 0.090 inches. Furthermore, in some embodiments, the distal portion of the working lumen defines a second inner diameter greater than or equal to 0.087 inches and less than or equal to 0.090 inches.

The working lumen may define an inner diameter extending from the proximal end to the distal end. In some embodiments, the inner diameter is approximately equal to 0.088 inches. The elongated sheath may define a working length approximately equal to 95 centimeters. In some embodiments, the proximal portion of the working lumen may equal approximately 85 centimeters in length, and the distal portion of the working lumen may equal approximately 10 centimeters in length.

In some embodiments, the working lumen defines an inner diameter greater than or equal to 0.088 inches. In some embodiments, the inner diameter of the working lumen is substantially equal to 0.091 inches.

In some embodiments, the first outer diameter is approximately equal to 0.137 inches, and the second outer diameter is approximately equal to 0.124 inches such that the elongated sheath fits through a 10 Fr opening. As well, in some embodiments, the proximal portion of the working lumen defines a first inner diameter greater than or equal to 0.101 inches and less than or equal to 0.113 inches, and the distal portion of the working lumen defines a second inner diameter greater than or equal to 0.101 inches and less than or equal to 0.113 inches. In some embodiments, the first inner diameter is approximately equal to 0.113 inches and the second inner diameter is approximately equal to 0.101 inches.

In some embodiments, the elongated sheath defines a working length that is long enough to enable the distal end to reach at least a cervical portion of a patient's internal carotid artery from the femoral artery. In some embodiments, the elongated sheath defines a working length that is long enough to enable the distal end to reach at least a petrous portion of a patient's internal carotid artery from the femoral artery. The working length may also be long enough to enable the distal end to reach at least a cavernous portion of a patient's internal carotid artery from the femoral artery.

The balloon may extend around and beyond a distal tip of the elongated sheath and define a funnel-shaped opening into the distal port when the balloon is in an inflated state such that the balloon does not occlude the working lumen of the distal port. Additionally, in some embodiments, the elongated sheath is arranged and configured to have sufficient stiffness and tip flexibility to enable insertion of the working length of the sheath into a patient's vasculature through an arteriotomy in the patient's carotid artery to position the distal port at a target site in at least one of a petrous portion of a patient's internal carotid artery, a cavernous portion of a patient's internal carotid artery, and a cerebral portion of a patient's internal carotid artery.

The disclosure also includes a balloon guiding sheath that includes an elongated sheath having a proximal end, a distal end, a proximal portion defining a first outer diameter, and a distal portion defining a second outer diameter; an access port located on the proximal end; a distal port located on the distal end; a working lumen extending through the elongated sheath between the access port and the distal port; an inflation port on the proximal end; an inflatable balloon coupled to the distal end; and an inflation lumen extending through the elongated sheath between the inflation port and the balloon, the inflation lumen comprising a distal inflation port extending through an endwall of the elongated sheath, wherein the inflation lumen is not in fluid communication with the working lumen between the access port and the distal port. In such embodiments, the elongated sheath is sized and configured to enable direct insertion into a patient's vasculature through an arteriotomy in the patient's femoral artery to position the balloon at a target site.

In some embodiments, the balloon guiding sheath includes a first inflation hole extending from the inflation lumen through a sidewall of the elongated sheath, wherein when a guide wire is inserted into the inflation lumen and out through the distal inflation port, the distal inflation port thereby creates a seal against the guide wire. The inflation lumen may thereby be arranged and configured to enable flow of at least one of fluid and media through the inflation lumen into the first inflation hole and into the balloon to thereby inflate the balloon.

In some embodiments, the inflation lumen is a first inflation lumen, and the balloon guiding sheath further includes a second inflation lumen extending through the elongated sheath between the inflation port and the balloon, the second inflation lumen comprising a second distal inflation port extending through the endwall of the elongated sheath, wherein the second inflation lumen is not in fluid communication with the working lumen between the access port and the distal port; and a second inflation hole extending from the second inflation lumen through the sidewall of the elongated sheath, wherein when a second guide wire is inserted into the second inflation lumen and out through the second distal inflation port, the second distal inflation port thereby creates a seal against the second guide wire. The second inflation lumen may be arranged and configured to enable flow of at least one of fluid and media through the second inflation lumen into the second inflation hole and into the balloon to thereby inflate the balloon.

The elongated sheath may define a central axis extending from the proximal end to the distal end, wherein at least a portion of the working lumen overlaps the central axis of the elongated sheath, and wherein the inflation lumen does not overlap the central axis of the elongated sheath. In some embodiments, the elongated sheath does not have a generally constant outer diameter along its working length.

Even still, in some embodiments, the elongated sheath defines a working length long enough to enable the distal end to reach at least a cervical portion of a patient's internal carotid artery from the carotid artery. In some embodiments, the working length is long enough to enable the distal end to reach a cavernous portion of the patient's internal carotid artery from the femoral artery.

The elongated sheath may be arranged and configured to have sufficient stiffness and tip flexibility to enable insertion of the working length of the sheath into a patient's vasculature through an arteriotomy in the patient's carotid artery to position the distal port at a target site in at least one of a petrous portion of a patient's internal carotid artery, a cavernous portion of the patient's internal carotid artery, and a cerebral portion of the patient's internal carotid artery.

The disclosure also includes a method for using a balloon guiding sheath comprising inserting the guiding sheath directly into a patient's vasculature through an arteriotomy in a patient's femoral artery; advancing the guiding sheath through the patient's vasculature and positioning the distal end in a target site of a patient's internal carotid artery; and/or inflating the balloon via the inflation lumen. In some embodiments, the target site is a cervical portion of the internal carotid artery. Yet, in some embodiments, the target site is a petrous portion of the internal carotid artery. Even still, in some embodiments, the target site is a cavernous portion of the internal carotid artery.

In some embodiments, the method includes applying relatively low pressure to the access port to suction an embolus; deflating the balloon; and/or withdrawing the guiding sheath through the arteriotomy in the carotid artery. In some embodiments, the method includes inserting a tool into the guiding sheath through the access port after positioning the distal end at the target site; advancing the tool through the guiding sheath; actuating the tool to retrieve an embolus; withdrawing the tool from the guiding sheath; deflating the balloon; and/or withdrawing the guiding sheath through the arteriotomy in the carotid artery.

In some embodiments, inflating the balloon via the inflation lumen occurs in response to: inserting a guide wire into the working lumen; and/or sealing an outer surface of the guide wire against an inner surface of the working lumen adjacent the distal port. Methods may include flowing at least one of fluid and media through a space within the inflation lumen between the guide wire and an inner surface of the inflation lumen and into the at least one inflation hole and into the balloon to thereby inflate the balloon.

In some embodiments, the method includes removing the guide wire from the working lumen. In response to removing the guide wire from the working lumen, methods may include deflating the balloon.

The disclosure also includes a balloon guiding sheath, comprising an elongated sheath having a proximal end, a distal end opposite the proximal end, an inner tube extending between the proximal end and the distal end, an outer tube surrounding the inner tube and extending between the proximal end and the distal end, an access port located adjacent the proximal end, a distal port located adjacent the distal end, and a working lumen extending through the elongated sheath between the access port and the distal port. In some embodiments, the balloon guiding sheath also includes an inflatable balloon located on an outer surface of the elongated sheath adjacent the distal end, the inflatable balloon being fluidly coupled to an inflation lumen extending between the inflatable balloon and an inflation port located adjacent the proximal end. Additionally, in some embodiments, the elongated sheath is sized and configured to enable direct insertion into a patient's vasculature through an arteriotomy in at least one of the patient's carotid artery and vertebral artery to position the balloon at a target site.

In some embodiments, the balloon guiding sheath further comprises a reinforcement layer located between the inner tube and the outer tube, the reinforcement layer arranged and configured to enable flow of at least one of fluid and media through the inflation lumen.

In some embodiments, the elongated sheath defines a working length comprising a proximal portion, a distal tip, and a distal portion located between the proximal portion and the distal tip. In some embodiments, the working length is about 90 centimeters. In some embodiments, the working length is about 95 centimeters. In some embodiments, the working length is about 100 centimeters. In some embodiments, the working length is less than 90 centimeters (e.g., 85 centimeters). In some embodiments, the working length is greater than 100 centimeters (e.g., 105 centimeters).

In some embodiments, the elongated sheath defines a working length long enough to enable the distal port to reach at least a cervical portion of a patient's internal carotid artery from the carotid artery. In some embodiments, the working length is long enough to enable the distal port to reach a cavernous portion of the patient's internal carotid artery from the carotid artery. In some embodiments, the elongated sheath is arranged and configured to have sufficient stiffness and tip flexibility to enable insertion of the working length of the sheath into a patient's vasculature through an arteriotomy in the patient's carotid artery to position the distal port at a target site in at least one of a petrous portion of a patient's internal carotid artery, a cavernous portion of the patient's internal carotid artery, and a cerebral portion of the patient's internal carotid artery.

In some embodiments, the elongated sheath defines a generally constant outer diameter from the proximal portion to the distal portion. In some embodiments, the generally constant outer diameter is equal to 0.123 inches. In some embodiments, the generally constant outer diameter is equal to 0.110 inches. In some embodiments, the generally constant outer diameter is equal to 0.102 inches.

In some embodiments, the elongated sheath defines a generally constant inner diameter from the proximal portion to the distal portion. In some embodiments, the generally constant inner diameter is equal to 0.103 inches. In some embodiments, the generally constant inner diameter is equal to 0.088 inches. In some embodiments, the generally constant inner diameter is equal to 0.087 inches.

In some embodiments, the elongated sheath defines an outer diameter that tapers downward from the proximal portion to the distal portion.

In some embodiments, the inflatable balloon is located in a non-recessed portion of the outer surface.

Other objects and advantages of the embodiments herein will become readily apparent from the following detailed description taken in conjunction with the accompanying drawings. The embodiments described above include many optional features and aspects. Features and aspects of the embodiments can be combined.

### BRIEF DESCRIPTION OF THE DRAWINGS

These and other features, aspects, and advantages are described below with reference to the drawings, which are intended to illustrate, but not to limit, the invention. In the drawings, like reference characters denote corresponding features consistently throughout similar embodiments.

FIG. 1A illustrates a balloon guiding sheath, according to some embodiments.

FIGS. 1B and 1C illustrate a cross-sectional view of section 1-1 of the balloon guiding sheath, with the balloon in a deflated state and an inflated state, respectively according to some embodiments.

FIG. 2 illustrates a cross-sectional view of section 2-2 of the balloon guiding sheath, according to some embodiments.

FIG. 3 illustrates a cross-sectional view of section 3-3 of the balloon guiding sheath, according to some embodiments.

FIG. 4 illustrates a cross-sectional view of section 2-2 of the balloon guiding sheath, according to some embodiments.

FIG. 5 illustrates a working length and generally constant outer diameter of the elongated sheath, according to some embodiments.

FIG. 6 illustrates an anatomy of an internal carotid artery, according to some embodiments.

FIGS. 7A and 7B illustrate cross-sectional views of section 7-7 of the balloon guiding sheath with the balloon in a deflated state and an inflated state, respectively, according to some embodiments.

FIG. 8 illustrates another balloon guiding sheath, according to some embodiments.

FIG. 9 illustrates a cross-sectional view of section 9-9 of the balloon guiding sheath, according to some embodiments.

FIG. 10 illustrates a cross-sectional view of section 10-10 of the balloon guiding sheath, according to some embodiments.

FIGS. 11A and 11B illustrate cross-sectional views of section 11-11 of the balloon guiding sheath with the balloon in a deflated state and an inflated state, respectively, according to some embodiments.

FIGS. 12A and 12B illustrate cross-sectional views of section 12-12 of the balloon guiding sheath with the balloon in a deflated state and an inflated state, respectively, according to some embodiments.

FIGS. 13A, 13B, and 13C illustrate cross-sectional views of section 13-13 of the balloon guiding sheath, according to some embodiments.

FIG. 14A illustrates another balloon guiding sheath, according to some embodiments.

FIG. 14B illustrate cross-sectional views of section 14B-14B of the balloon guiding sheath, according to some embodiments.

FIGS. 15, 16, 17, and 18 illustrate methods of using a balloon guiding sheath, according to some embodiments.

FIG. 19 illustrates another balloon guiding sheath, according to some embodiments.

FIGS. 20A and 20B illustrate cross-sectional views of section 20-20 of the balloon guiding sheath, according to some embodiments.

FIG. 21 illustrates a cross-sectional view of section 21-21 of the balloon guiding sheath, according to some embodiments.

FIG. 22 illustrates yet another balloon guiding sheath, according to some embodiments.

FIGS. 23A and 23B illustrate cross-sectional views of section 23-23 of the balloon guiding sheath, according to some embodiments.

FIG. 24 illustrates a cross-sectional view of section 24-24 of the balloon guiding sheath, according to some embodiments.

FIGS. 25, 26, and 27 illustrate methods of using a balloon guiding sheath, according to some embodiments.

FIG. 28 illustrates a cross-sectional view of a balloon guiding sheath, with the balloon in an inflated state, according to some embodiments.

### DETAILED DESCRIPTION

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Additionally, reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

### LIST OF REFERENCE NUMERALS

10—Balloon guiding sheath
12—Elongated sheath
13a, c—Inner tube
13b, d—Outer tube
14—Access port
16—Inflation port
18—Inflatable balloon
20—Distal port
21—Distal inflation port
22—Working lumen
30—First inflation lumen
31—Reinforcement layer
32—Second inflation lumen
34—Outer diameter
36—Inner diameter
38—Working length
39—Generally constant outer diameter
50—Internal carotid artery
52—Cervical portion
54—Petrous portion
56—Cavernous portion
58—Cerebral portion
59—Distal tip
60—Central axis
70—First portion
72—First inner diameter
74—Second portion
76—Second inner diameter
80—First inflation hole
82—Second inflation hole
84—Guide wire
90—Proximal portion
92—First outer diameter
94—Distal portion
96—Second outer diameter
98—Middle portion
110—Proximal inflation lumen
112—First inner diameter
114—Distal inflation lumen
116—Second inner diameter

Baby BOSS Multiple Tube Embodiments

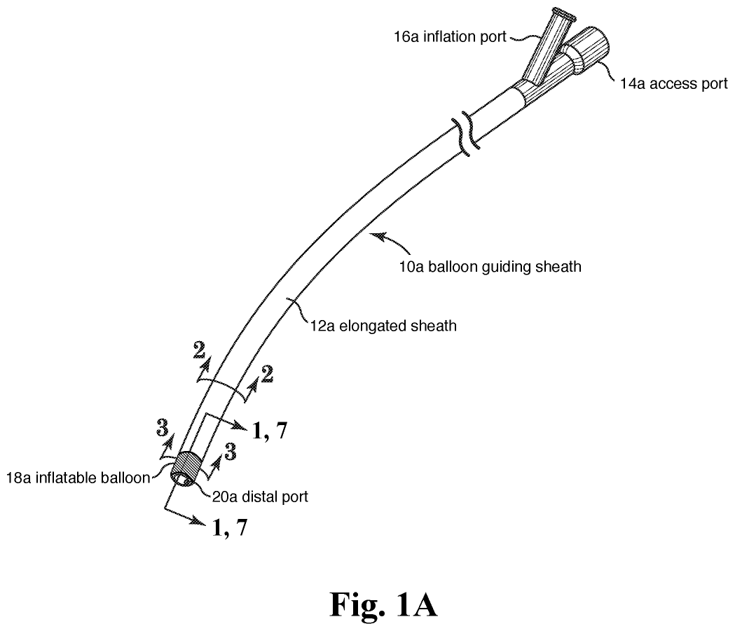

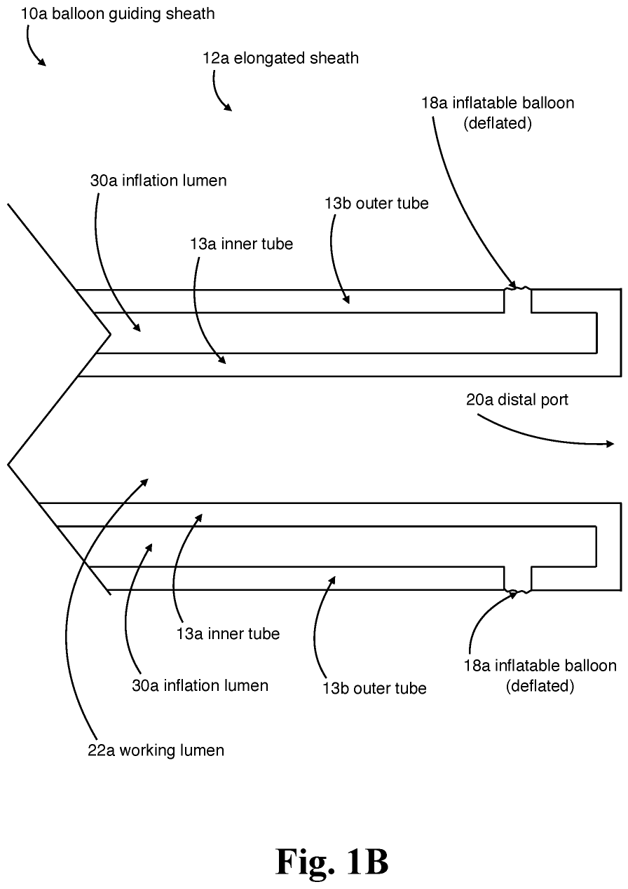

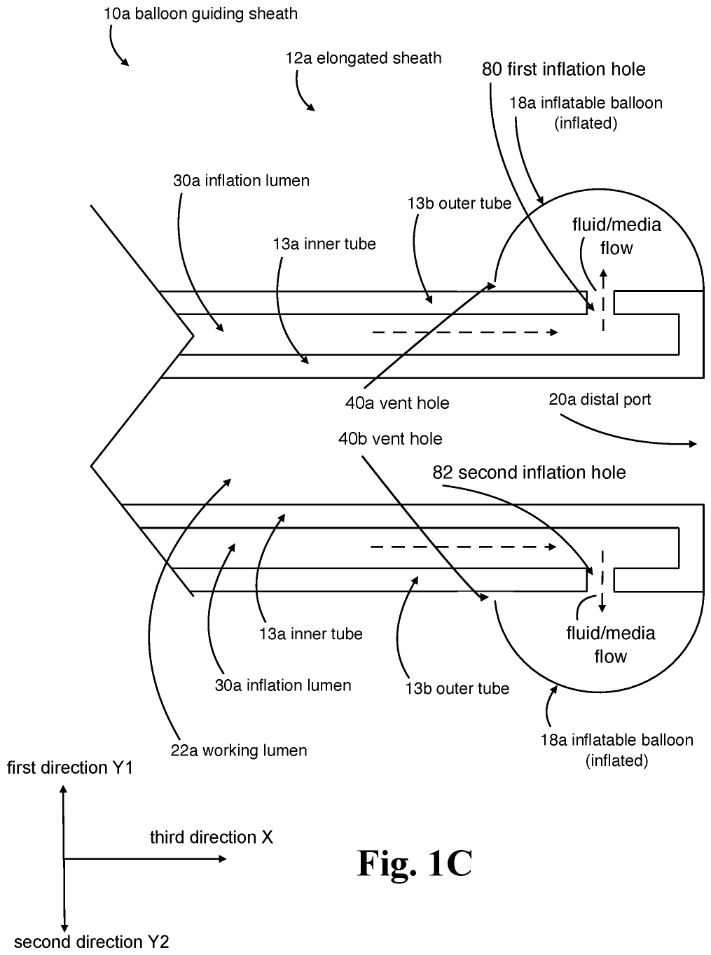

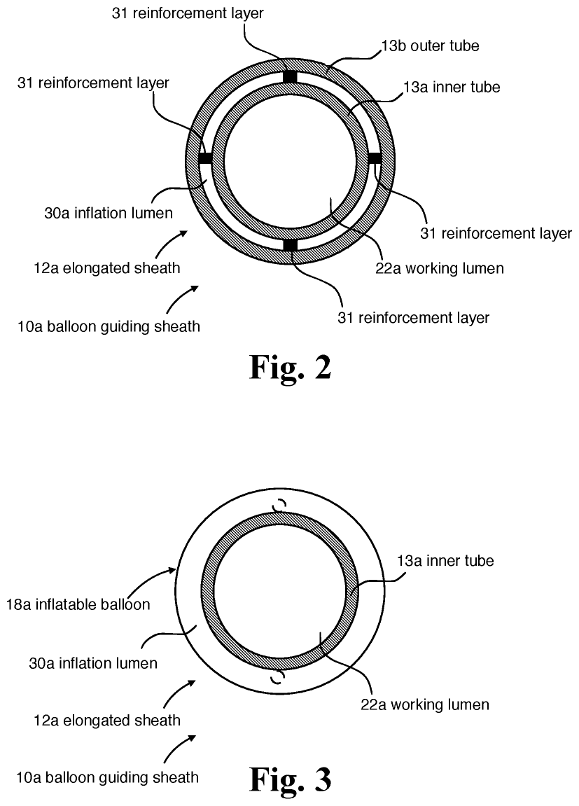

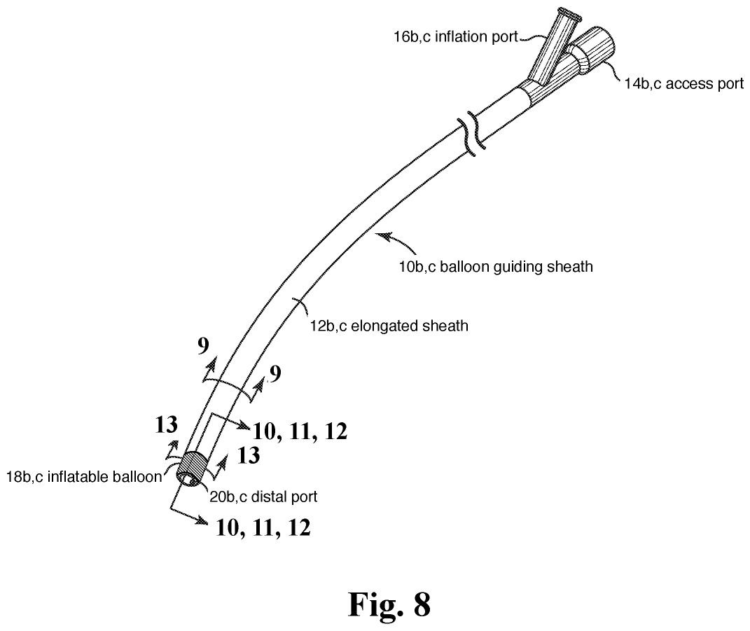

A balloon guiding sheath 10a in accordance with embodiments of the invention can be described generally with reference to FIGS. 1A-7. As shown in FIG. 1A, a balloon guiding sheath 10a may comprise an elongated sheath 12a having a proximal end and a distal end. With reference to FIG. 2, the elongated sheath 12a may include an inner tube 13a and an outer tube 13b that surrounds the inner tube 13a. It should be appreciated that many embodiments may also be implemented with one tube, as will be discussed later regarding FIG. 8. Other embodiments may include three or more tubes, also referred to as layers.

The components of the balloon guiding sheath 10 may be formed from a polymer (e.g. polytetrafluoroethylene, nylon, and the like). In some embodiments, the components may comprise Pellethane 63D or higher. Generally, the material selection may be focused on enhancing pushability in the balloon guiding sheath 10 as opposed to flexibility. However, it should be appreciated that in some embodiments the material selection may be focused on either or both pushability and/or flexibility.

As shown in FIG. 1A, the guiding sheath 10a may include an access port 14a located on the proximal end and an inflation port 16a also located on the proximal end. The guiding sheath 10a may include an inflatable balloon 18a coupled to the distal end and a distal port 20a also located on the distal end. As shown in FIGS. 2 and 3, the guiding sheath 10a may include a working lumen 22a extending through the elongated sheath 12 between the access port 14a and the distal port 20a.

The guiding sheath 10a may also include an inflation lumen 30a that extends between the inflation port 16a and the balloon 18a. In the embodiment disclosed in FIGS. 1-7, the inflation lumen 30a is located between the inner tube 13a and the outer tube 13b. As illustrated, the inflation lumen 30a is not in fluid communication with the working lumen 22a. However, as will be discussed with regards to FIGS. 8-11B, embodiments may be arranged and configured whereby the working lumen 22 is in fluid communication with the inflation lumen 30.

Now with reference to FIGS. 1B and 1C, a cross-sectional side view of section 1-1 is illustrated. As shown, the balloon 18a may move between a deflated state (FIG. 1B) and an inflated state (FIG. 1C) in response to fluid and/or media traveling through the inflation lumen 30a and into the balloon 18a. In such embodiments, the inflation lumen 30a is not in fluid communication with working lumen 22a. In this regard, the balloon 18a may be arranged and configured to inflate and deflate irrespective of any interaction with the working lumen 22a.

With reference to FIG. 2, the guiding sheath 10a may include a reinforcement layer 31 located between the inner tube 13a and the outer tube 13b. The reinforcement layer 31 may be arranged and configured to enable flow of at least one of fluid and media through the inflation lumen 30a to thereby inflate the balloon 18a. The reinforcement layer 31 may be comprised of coiled and/or braided strands of material (e.g. stainless steel or polymer wire).

The elongated sheath 12a may be sized and configured to enable direct insertion into a patient's vasculature through an arteriotomy in the patient's carotid artery and/or vertebral artery. During use, the guiding sheath 10a may be positioned at a target site, whereby the balloon 18a is inflated to occlude blood flow through the patient's artery. The working lumen 22a, via the access port 14a, shall be arranged and configured to receive various instrumentation, such as a guide wire, tool(s), and the like. The instrumentation is then advanced through the working lumen 22a to the target site to treat and remove the embolus.

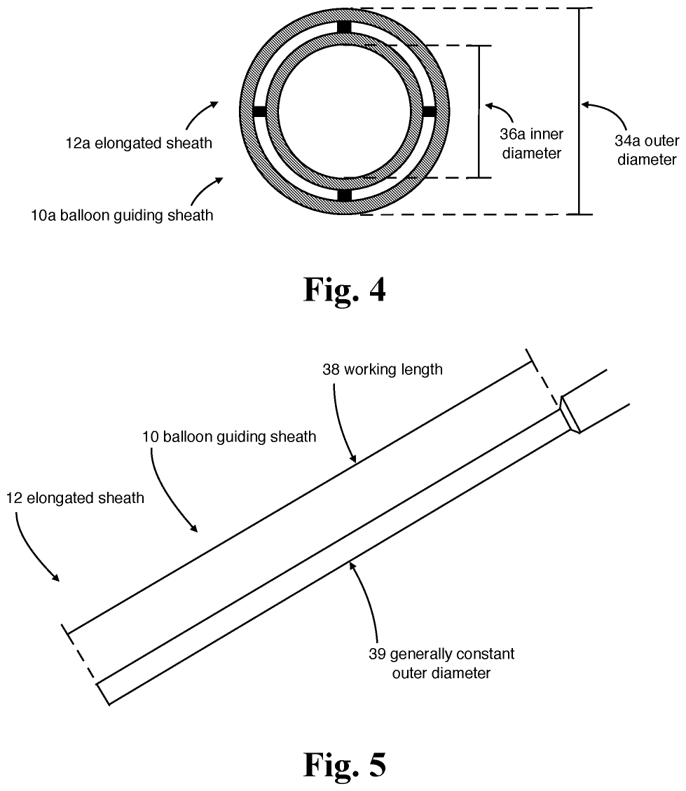

Now with reference to FIG. 4, the elongated sheath 12a may define an outer diameter 34a that is less than or equal to 0.104 inches. In this regard, the elongated sheath 12a may fit through an 8 Fr opening. It should be appreciated that the opening may be a puncture, cavity, and/or aperture whether in the patient's vasculature or in any medical device used to treat the embolus. Moreover, the working lumen 22a may define an inner diameter 36a less than or equal to 0.090 inches. In some embodiments, the inner diameter 36a of the working lumen 22a is greater than or equal to 0.087 inches. However, it should be appreciated that the outer and inner diameters 34a, 36a may define any such dimension. For example, the outer diameter 34a may be greater than or equal to 0.104 inches. Additionally, in some embodiments, the inner diameter 36a may be less than or equal to 0.088 inches, or greater than or equal to 0.090 inches.

To further elaborate on the various outer and inner diameter 34, 36 sizes, in some embodiments, the inner diameter 36a is greater than 0.088 inches. For example, the inner diameter 36a may be substantially equal to 0.091 inches. In some embodiments, the inner diameter 36a is substantially equal to 0.103 inches. In some embodiments, the inner diameter 36a is substantially equal to 0.110 inches. In some embodiments, the inner diameter 36a is substantially equal to 0.120 inches. Generally, it should be appreciated that the inner diameter 36a may define any size diameter configured to perform thrombectomy or embolectomy in the internal carotid artery and other vessels of a patient.

Furthermore, in some embodiments, the outer diameter 34 is greater than or equal to 0.105 inches. More specifically, in some embodiments, the outer diameter 34 is substantially equal to 0.115 inches such that the elongated sheath fits through a 9 Fr Pinnacle sheath sold by Terumo Interventional Systems (www.terumois.com). Additionally, the outer diameter 34 may fit through a customized 8 Fr sheath whereby the sheath includes a coiled inner sheath that is laminated. The customized sheath may also include a braided outer layer for added stiffness, which is further described in this disclosure in various locations.

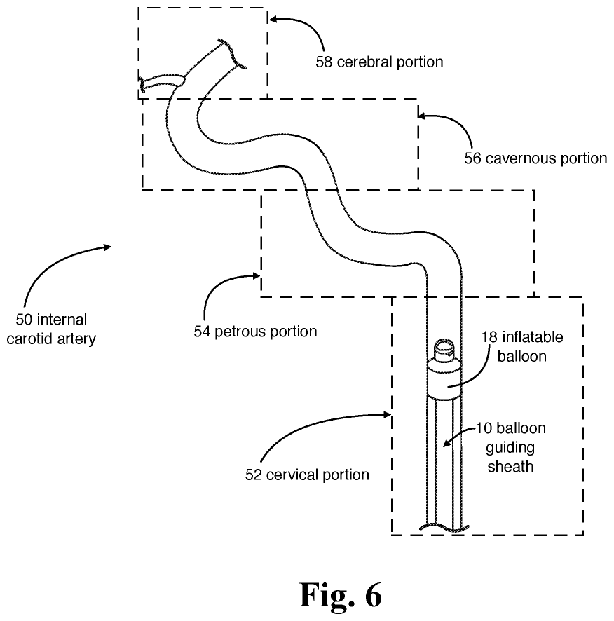

As shown in FIG. 5, the elongated sheath 12a may have a generally constant outer diameter 39 along its working length 38a. However, some embodiments may have varying diameters along the working length 38. With additional reference to FIG. 6, the elongated sheath 12a may have a working length 38a that is long enough to enable the distal end to reach at least a cervical portion 52 of a patient's internal carotid artery 50 from the carotid artery. Even still, in some embodiments, the working length 38a may be long enough to enable the distal end to reach a petrous portion 54, cavernous portion 56, and/or a cerebral portion 58 of a patient's internal carotid artery 50 from the carotid artery. It should be appreciated that the location of the dashed boxes in FIG. 6 are not exact and merely intended to distinguish between the various portions of the patient's internal carotid artery.

Generally, the guiding sheath 10 disclosed herein is intended to maximize the inner diameter 36a, while maintaining a relatively thin outer diameter 34a. This may result in overall less inflation area within the inflation lumen 30 to inflate the balloon 18. Because inflation time is directly related to inflation area and length of the inflation lumen 30, the working length 38 shall be less than or equal to 30 centimeters. However, in some embodiments, the working length 38 is greater than or equal to 30 centimeters.

To effectively reach various portions of the patient's internal carotid artery, the elongated sheath 12 may be arranged and configured to have sufficient stiffness and tip flexibility to enable insertion of the working length 38 of the elongated sheath 12 into a patient's vasculature through an arteriotomy in the patient's carotid artery. As such, the distal port 20a may be positioned at a target site in the petrous portion 54, cavernous portion 56, and the cerebral portion 58 of the patient's internal carotid artery 50.

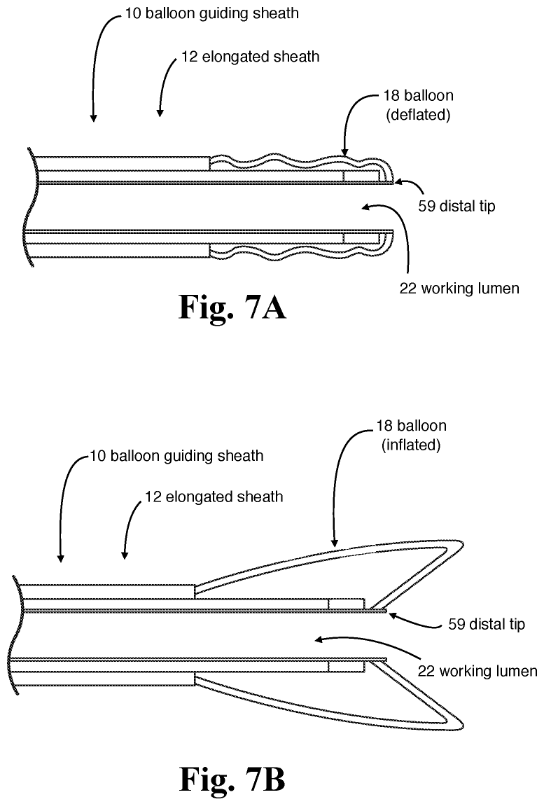

In order to safely remove the embolus from the target site, the balloon 18 may define various shapes and sizes. For example, as shown in FIGS. 7A and 7B, the balloon may be arranged and configured to extend around and beyond a distal tip 59 of the elongated sheath 12. With specific reference to FIG. 7B, the balloon 18 may define a funnel-shaped opening into the distal port 20 when the balloon 18 is in an inflated state. The funnel-shape may thereby ensure that the embolus and any harmful tissue is effectively directed and guided into the working lumen 22 for complete removal from the patient's artery. Furthermore, the funnel-shape may safeguard the balloon 18 so that the balloon 18 does not occlude the working lumen 22 and any instrumentation or tissue that needs to travel through the working lumen 22.

Generally, the guiding sheath 10 disclosed herein may be implemented with any size, shape, and location of balloon 18. For example, in some embodiments, the balloon 18 does not extend beyond the distal tip 59 of the working lumen 22.

Baby BOSS Tip-Occluding Embodiments

Another balloon guiding sheath 10b in accordance with embodiments of the invention is now described with reference to FIGS. 8-13C. As shown in FIG. 8, the guiding sheath 10b includes an elongated sheath 12b having a proximal end and a distal end. The guiding sheath 10b may include an access port 14b located on the proximal end and an inflation port 16b on the proximal end. The guiding sheath 10b may include an inflatable balloon 18b coupled to the distal end and a distal port 20b located on the distal end. The elongated sheath 12b may be sized and configured to enable direct insertion into a patient's vasculature through an arteriotomy in at least one of the patient's carotid artery and vertebral artery to position the balloon 18b at the target site of the embolus.

Similar to embodiment 10a, the guiding sheath 10b may also include a working lumen 22b extending through the elongated sheath 12b between the access port 14b and the distal port 20b. The guiding sheath 10b may include an inflation lumen 30b extending through the elongated sheath 12b between the inflation port 16b and the balloon 18b. Unlike embodiment 10a, guiding sheath 10b may be arranged and configured such that the working lumen 22b transitions between being in fluid communication with the inflation lumen 30b and then not being in fluid communication with the inflation lumen 30b. In this regard, prior to advancing the guide wire 84 through the working lumen 22b, the working lumen 22b and the inflation lumen 30b are in fluid communication with each other. However, once the guide wire 84 is advanced through the working lumen 22b and beyond the distal tip 59 of the elongated sheath 12b, the guide wire 84 thereby occludes the distal tip 59 which cuts off the fluid communication between the working lumen 22b and the inflation lumen 30b thereby allowing fluid and/or media to flow through the inflation lumen 30b and into the balloon 18b to inflate the balloon 18b.

Stated differently, the working lumen 22b may not be in fluid communication with the inflation lumen 30b when the balloon 18b is inflated. Additionally, the working lumen 22b may be in fluid communication with the inflation lumen 30b when the balloon 18b is at least partially deflated. However, either scenario may apply if the balloon 18b is in the midst of inflating or deflating. In other words, it can be said that the working lumen 22b is not in fluid communication with the inflation lumen 30b when the balloon 18b is inflated or at least partially inflated. Likewise, the working lumen 22b is in fluid communication with the inflation lumen 30b when the balloon 18b is deflated or at least partially deflated. Such tip-occluding embodiments may be beneficial because they maximize the inner diameter of the working lumen 22b while minimizing the outer diameter of the elongated sheath 12b.

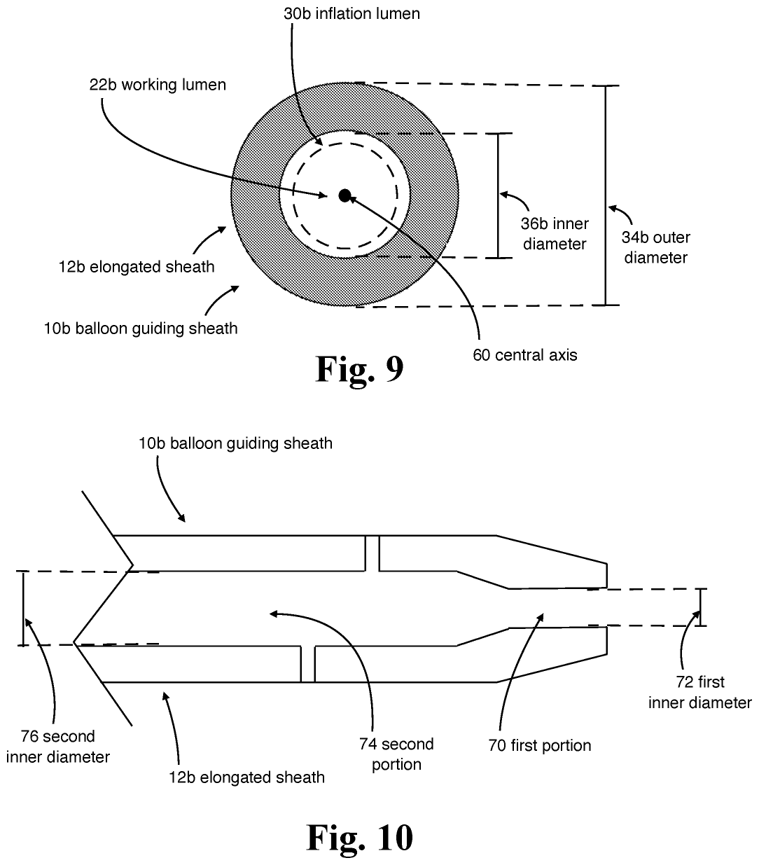

To further describe the relationship between the working lumen 22b and the inflation lumen 30b we now refer to FIG. 9. The elongated sheath 12b may define a central axis 60 extending from the proximal end to the distal end. As shown in FIG. 9, at least a portion of the working lumen 22b may overlap the central axis 60 of the elongated sheath 12b, while the inflation lumen 30b does not overlap the central axis 60 of the elongated sheath 12b.

With continued reference to FIG. 9, the elongated sheath 12b may define an outer diameter 34b less than or equal to 0.104 inches such that the elongated sheath 12b fits through an 8 Fr opening. Additionally, the working lumen 22b may define an inner diameter 36b less than or equal to 0.090 inches. In some embodiments, the inner diameter 36b of the working lumen 22b is greater than or equal to 0.087 inches. Similar to above, it should be appreciated that the outer and inner diameters 34b, 36b may define any such dimension. For example, the outer diameter 34b may be greater than or equal to 0.104 inches. Additionally, in some embodiments, the inner diameter 36b may be less than or equal to 0.087 inches, or greater than or equal to 0.090 inches.

Similar to the embodiment described above, the elongated sheath 12b also has a generally constant outer diameter 39 along its working length 38b. Additionally, the elongated sheath 12b may also define a working length 38b long enough to enable the distal end to reach at least a cervical portion 52, petrous portion 54, cavernous portion 56, and a cerebral portion 58 of the patient's internal carotid artery 50 from the carotid artery.

Furthermore, the balloon 18b may extend around and beyond a distal tip 59 of the elongated sheath 12b and define a funnel-shaped opening into the distal port 20b when the balloon 18b is in an inflated state. Moreover, the elongated sheath 12b may also be arranged and configured to have sufficient stiffness and tip flexibility to enable insertion of the working length 38b of the elongated sheath 12b into a patient's vasculature through an arteriotomy in the patient's carotid artery to position the distal port 20b at a target site in at least one of the petrous portion 54, cavernous portion 56, and the cerebral portion 58 of the patient's internal carotid artery 50.

Now with reference to FIG. 10, the working lumen 22b may define various portions having different size diameters. As shown, the working lumen 22b may comprise a first portion 70 defining a first inner diameter 72 and a second portion 74 defining a second inner diameter 76. As shown, the second portion 74 is located proximal to the first portion 70. The second inner diameter 76 may be greater than the first inner diameter 72.

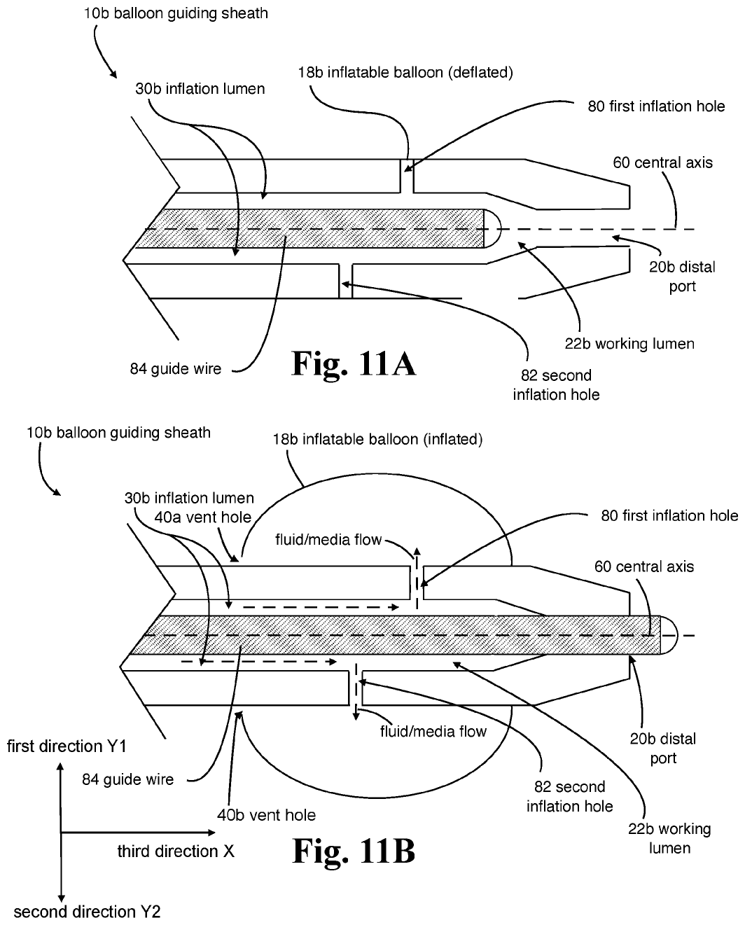

As shown in FIGS. 11A and 11B, the balloon guiding sheath 10b may include a first inflation hole 80 extending from the working lumen 22b through a sidewall of the elongated sheath 12b. In this regard, when a guide wire 84 is inserted into the working lumen 22b and out through the distal port 20b, the distal port 20b thereby creates a seal against the guide wire 84. Once the guide wire 84 creates a seal with the distal port 20b, this enables flow of fluid and/or media through the inflation lumen 30b into the first inflation hole 80 and into the balloon 18b to thereby inflate the balloon 18b. While many of the figures show the balloon 18 disposed flush with the distal port 20, it should be appreciated that the balloon 18 may be offset by any distance, as shown in FIGS. 11A and 11B. In some embodiments, the balloon 18 is offset about 4 millimeters from the distal port 20.

Furthermore, the guiding sheath 10b may include a second inflation hole 82 extending from the working lumen 22b through the sidewall of the elongated sheath 12b. Again, once the guide wire 84 creates a seal with the distal port 20b, this enables flow of fluid and/or media through the inflation lumen 30b into the second inflation hole 82 and into the balloon 18b to thereby inflate the balloon 18b. As shown in FIGS. 11A and 11B, the first and second inflation holes 80, 82 may be horizontally offset from each other. However, in some embodiments, the first and second inflation holes 80, 82 are substantially horizontally aligned with each other. The inflation holes may be staggered to thereby inflate various portions of the balloon at different times.

Furthermore, while not shown, the working lumen 22 may define more than two portions having more two ore more different diameters. In this manner, the guide wire 84 may occlude various portions of the working lumen 22 thereby allowing one or more balloons 18 to inflate at specific intervals. Such configurations may be beneficial in treating and removing different types and sizes of emboli.

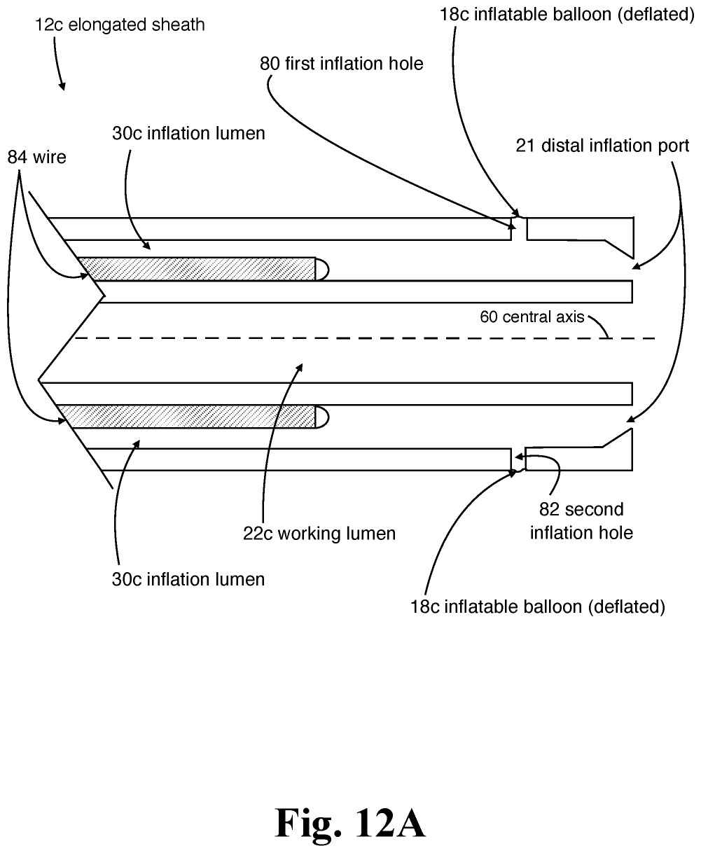

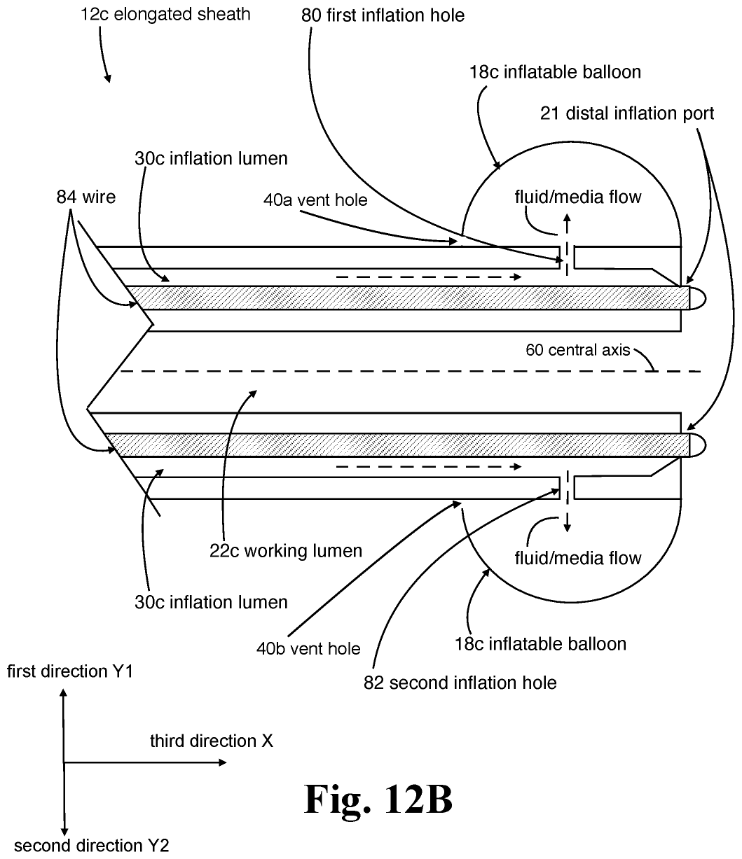

Now with reference to FIGS. 12A and 12B, the guiding sheath 10c may include one or more inflation lumen(s) 30c extending through the elongated sheath 12c between the inflation port 16c and the balloon 18c. As shown, the inflation lumen 30c may comprise a distal inflation port 21 extending through an endwall of the elongated sheath 12c. In this regard, the inflation lumen 30c is not in fluid communication with the working lumen between the access port 14c and the distal port 20c.

The balloon guiding sheath 10c may also include one or more inflation hole(s) 80, 82 extending from the inflation lumen 30c through a sidewall of the elongated sheath 12c. As such, when a guide wire 84 is inserted into the inflation lumen 30c and out through the distal inflation port 21, the distal inflation port 21 may thereby create a seal against the guide wire 84. Once the seal is created, the inflation lumen 30c may enable flow of at least one of fluid and media through the inflation lumen 30c into the one or more inflation hole(s) 80, 82 and into the balloon 18c to thereby inflate the balloon 18c, as shown in FIG. 12B. As further shown in FIGS. 12A and 12B, the elongated sheath 12c may have a generally constant outer diameter 39 along its working length.

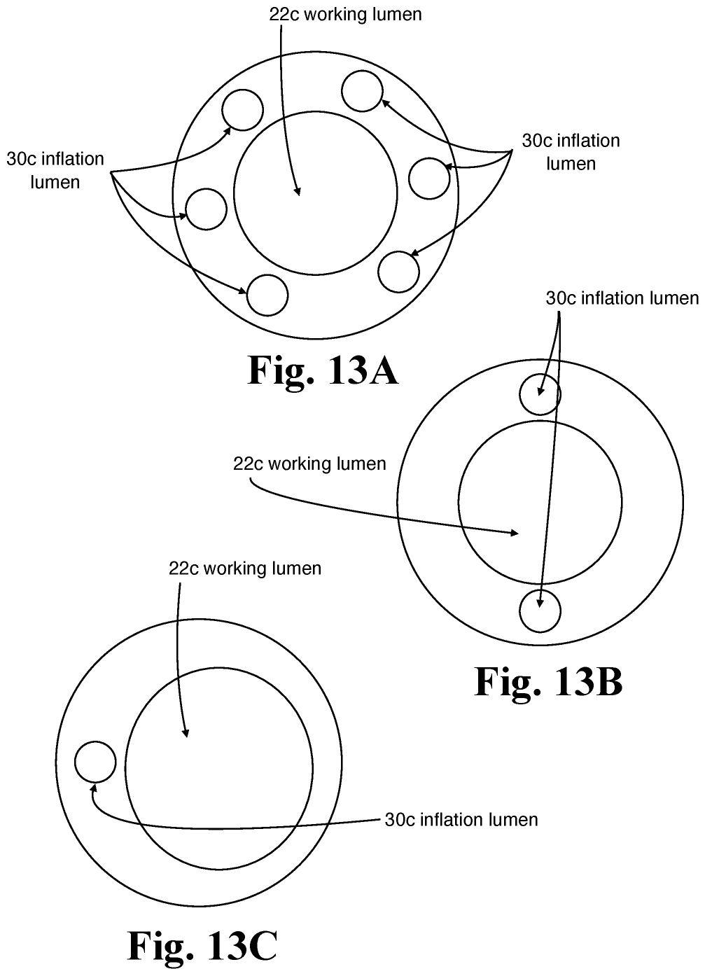

FIGS. 13A, 13B, and 13C illustrate a variety of cross-sectional views of section 13-13 of the elongated sheath 12c. As shown, the guiding sheath 10 may include one or more inflation lumen(s) 30c arranged in a variety of configurations. For example, as shown in FIG. 13A, the elongated sheath 12c may include six inflation lumens 30c arranged in any pattern around the working lumen 22c. As illustrated in FIG. 13B, the elongated sheath 12c may include two inflation lumens 30c arranged on opposite sides of the working lumen 22c. Even still, as shown in FIG. 13C, the elongated sheath 12c may include one inflation lumen 30c adjacent an oval-shaped working lumen 22c that is off-center with respect to the central axis. It should be appreciated that the working lumen 22c may define any such cross-sectional shape, such as circular, round, oblong, and even shapes such as triangular, rectangular, and any shape defining five or more sides.

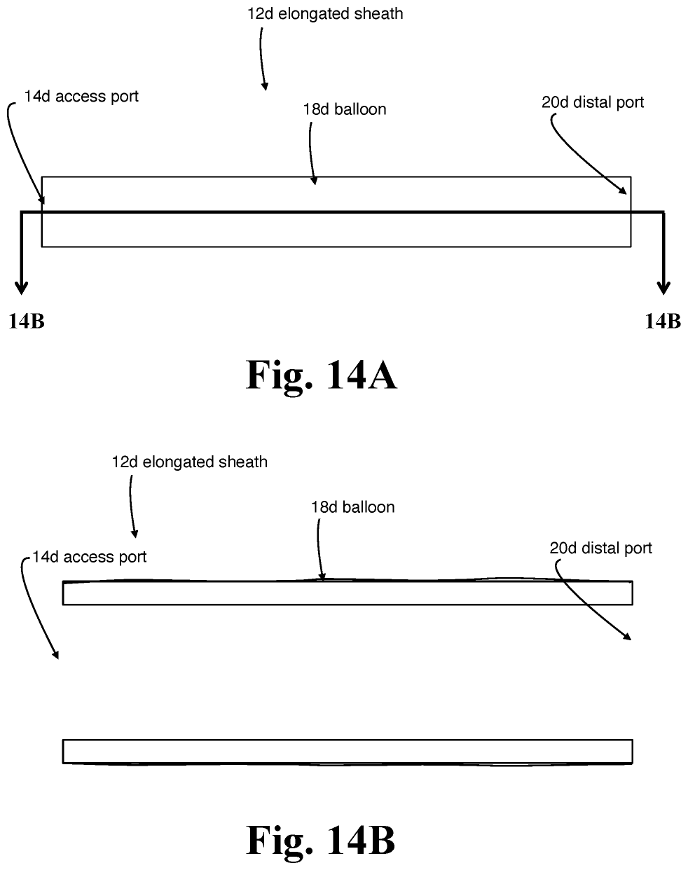

As shown in FIGS. 14A and 14B, the elongated sheath 12d may define another embodiment whereby the balloon 18d extends along the entire outer surface area, or at least more than half of the outer surface area, of the elongated sheath 12d. In such embodiments, the balloon 18d may extend all the way from the access port 14d to the distal port 20d. The balloon 18d material may be arranged and configured such that certain sections of the balloon may inflate/deflate at predetermined zones. Some embodiments, with respect to FIGS. 14A and 14B, may be devoid of inflation holes 80, 82.

Additionally, in some embodiments, the entire elongated sheath 12 is foldable, or self-expanding. This may allow the elongated sheath 12, while in it's folded state, to be moved to the target site and then expanded to thereby provide antegrade blood flow cessation.

All of the illustrated embodiments have shown the inflation holes 80, 82 to be disposed closer to the distal port 20 then the access port 14; however, many embodiments may be configured whereby the inflation holes 80, 82 are disposed closer to the access port 14 then the distal port 20. Such embodiments may result in shorter time to inflate and deflate the balloon 18.

Additionally, because of the need to minimize air in the balloon 18 during use, the guiding sheath 10 may be arranged and configured to include a vent hole(s) at the proximal end of the balloon 18. The vent hole(s) may be formed by bonding a wire between the balloon 18 and the elongated sheath 12, and then removing the wire prior to use. Thereby when the balloon 18 is inflated, the vent hole may allow air to escape through the vent hole, but not let fluid and/or media leak out. In this regard, the vent hole is large enough to allow air through, but small enough to prevent liquid and media from passing through.

Baby BOSS Method Embodiments

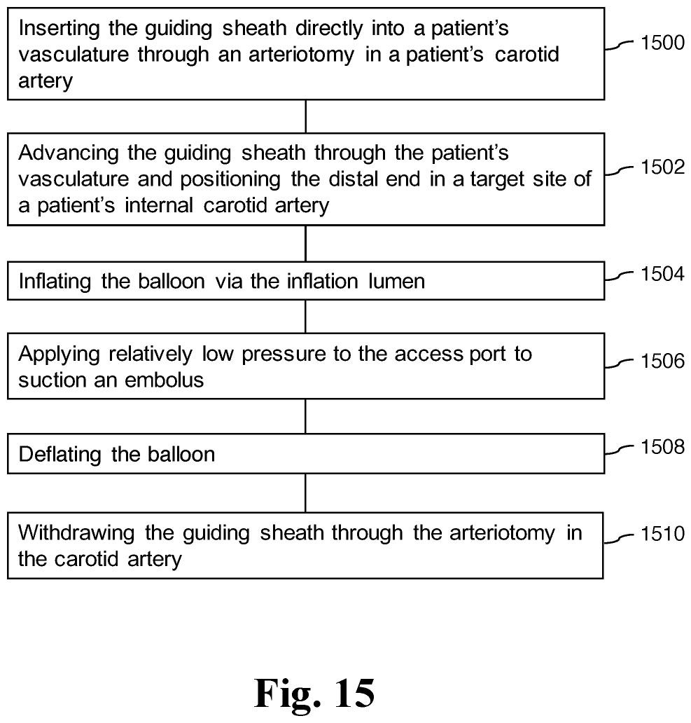

With reference to FIGS. 15-18, the disclosure also includes methods for using the balloon guiding sheaths 10a, 10b as described above. Some methods may be implemented with either guiding sheath 10a or 10b. However, some methods may only be implemented with embodiment 10b. Each circumstance will be described in detail below.

As shown in FIG. 15, methods may include inserting the guiding sheath 10a, 10b directly into a patient's vasculature through an arteriotomy in a patient's carotid artery (at step 1500). Methods may also include advancing the guiding sheath 10a, 10b through the patient's vasculature and positioning the distal end in a target site of a patient's internal carotid artery 50 (at step 1502). Once the guide sheath 10a, 10b has been advanced, methods may include inflating the balloon 18a, 18b via the inflation lumen 30a, 30b (at step 1504). As previously disclosed, the target site may be at least a cervical portion 52, petrous portion 54, cavernous portion 56, and a cerebral portion 58 of the internal carotid artery 50.

Furthermore, methods may include applying relatively low pressure to the access port 14a, 14b to suction an embolus (at step 1506). Methods may thereby include deflating the balloon 18a, 18b (at step 1508) and thereby withdrawing the guiding sheath 10a, 10b through the arteriotomy in the carotid artery (at step 1510).

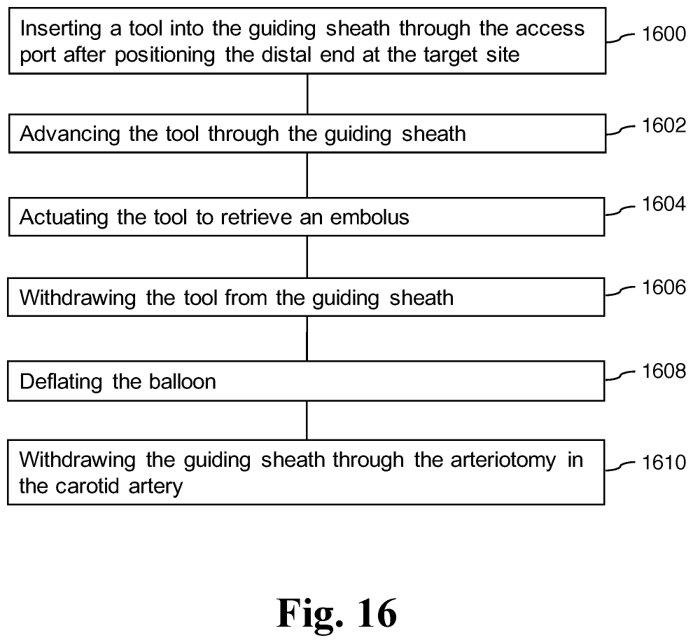

Now with reference to FIG. 16, after positioning the distal end at the target site, the user may insert a tool into the guiding sheath 10a, 10b through the access port 14a, 14b (at step 1600). The user may advance the tool through the guiding sheath 10a, 10b (at step 1602) and actuate the tool to retrieve the embolus (at step 1604). Once the embolus has been retrieved, the tool may be withdrawn from the guiding sheath 10a, 10b (at step 1606). In order to complete the embolus removal, the user may thereby deflate the balloon 18a, 18b (at step 1608) and withdraw the guiding sheath 10a, 10b through the arteriotomy in the carotid artery (at step 1610).

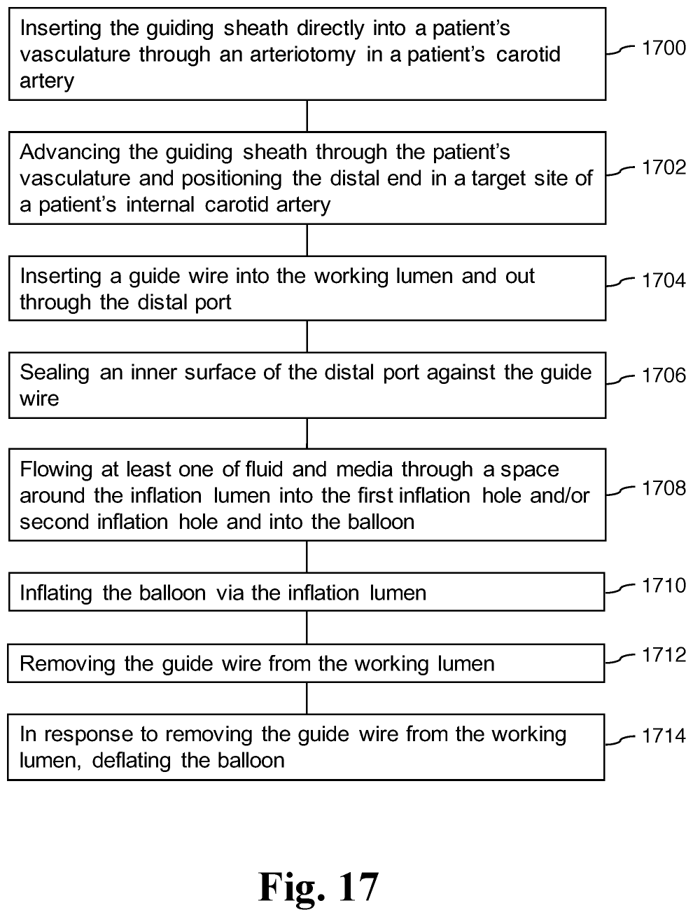

As shown in FIG. 17, the method may also include specific method steps to be performed with balloon guiding sheath 10b. In some embodiments, methods include inserting the guiding sheath 10b directly into a patient's vasculature through an arteriotomy in a patient's carotid artery (at step 1700). The user may then advance the guiding sheath 10b through the patient's vasculature and position the distal end in a target site of a patient's internal carotid artery (at step 1702).

In order to begin the balloon inflation process, the user may insert a guide wire 84 into the working lumen 22b and out through the distal port 20b (at step 1704) to thereby seal an inner surface of the distal port 20b against the guide wire 84 (at step 1706). Once the distal port 20b has been sealed, methods may include flowing fluid and/or media through a space around the inflation lumen 30b into the first inflation hole 80 and/or second inflation hole 82 and into the balloon 18b to inflate the balloon 18b (at step 1708). As the fluid and/or media flows into the balloon 18b via the inflation lumen 30b, the balloon 18b may become inflated (at step 1710) to occlude the artery.

Once the balloon 18b has been inflated, the user may perform none or any combination of steps 1600, 1602, 1604, and/or 1606 in order to remove the embolus. Upon completion of such steps, the user may remove the guide wire 84 from the working lumen 22b (at step 1712). As such, the working lumen 22b and the inflation lumen 30b may once again be in fluid communication, which means that the inflation lumen 30b is not able to adequately flow liquid and/or media into the balloon 18b to keep the balloon inflated. As such, in response to removing the guide wire 84 from the working lumen 22b, the balloon 18b may deflate (at step 1714).

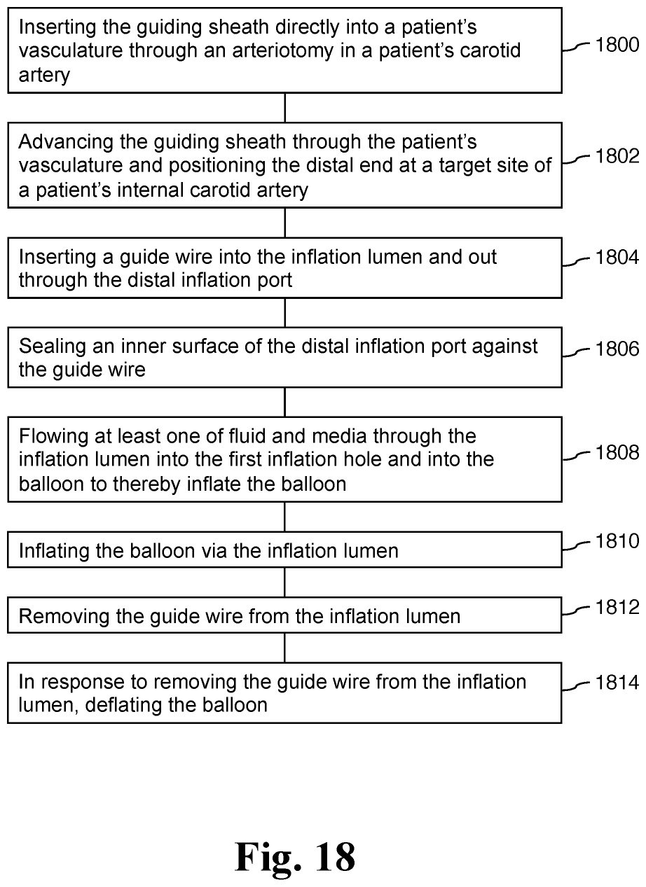

As illustrated in FIG. 18, methods may include steps for using a tip-occluding embodiment whereby the inflation lumen 30c is not in fluid communication with the working lumen 22c. Using such embodiments, methods may include inserting the guiding sheath 10c directly into a patient's vasculature through an arteriotomy in a patient's carotid artery (at step 1800). Methods may also include advancing the guiding sheath 10c through the patient's vasculature and positioning the distal end at a target site of a patient's internal carotid artery (at step 1802).

Once the guiding sheath 10c has been positioned in its desired location within the patient's carotid artery, methods may include inserting a guide wire 84 into the inflation lumen 30c and out through the distal inflation port 21 (at step 1804) and sealing an inner surface of the distal inflation port 21 against the guide wire 84 (at step 1806). Once the seal has been created, the method may include flowing at least one of fluid and media through the inflation lumen 30c into the first inflation hole 80 and into the balloon 18c to thereby inflate the balloon 18c (at step 1808) and thereby inflating the balloon 18c via the inflation lumen 30c (at step 1810).

In order to deflate the balloon 18c, methods may include removing the guide wire 84 from the inflation lumen 30c (at step 1812). In response to removing the guide wire 84 from the inflation lumen 30c, methods may include the step of deflating the balloon 18c (at step 1814).

BOSS Max and BOSS Slender Multiple Tube Embodiments

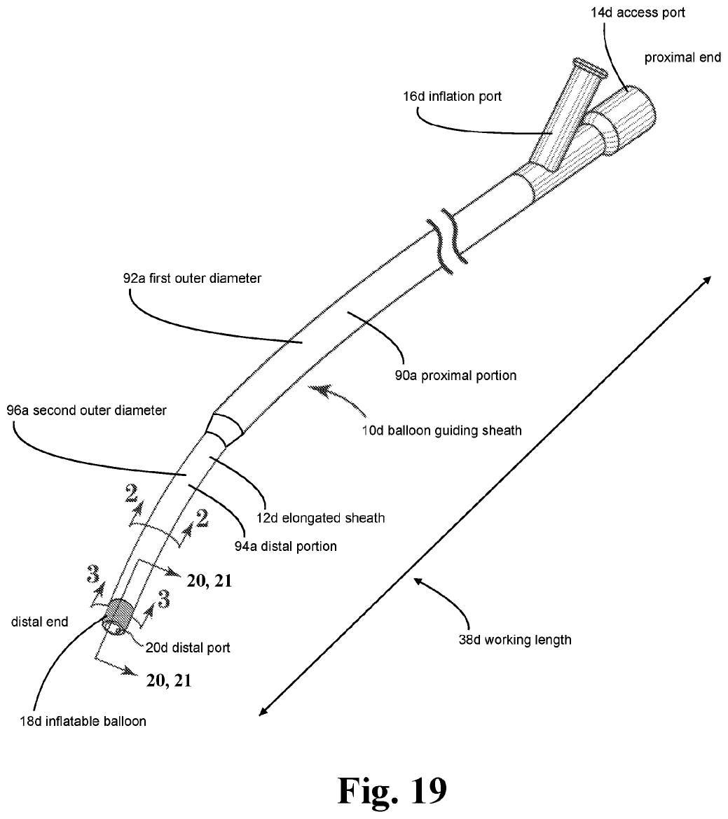

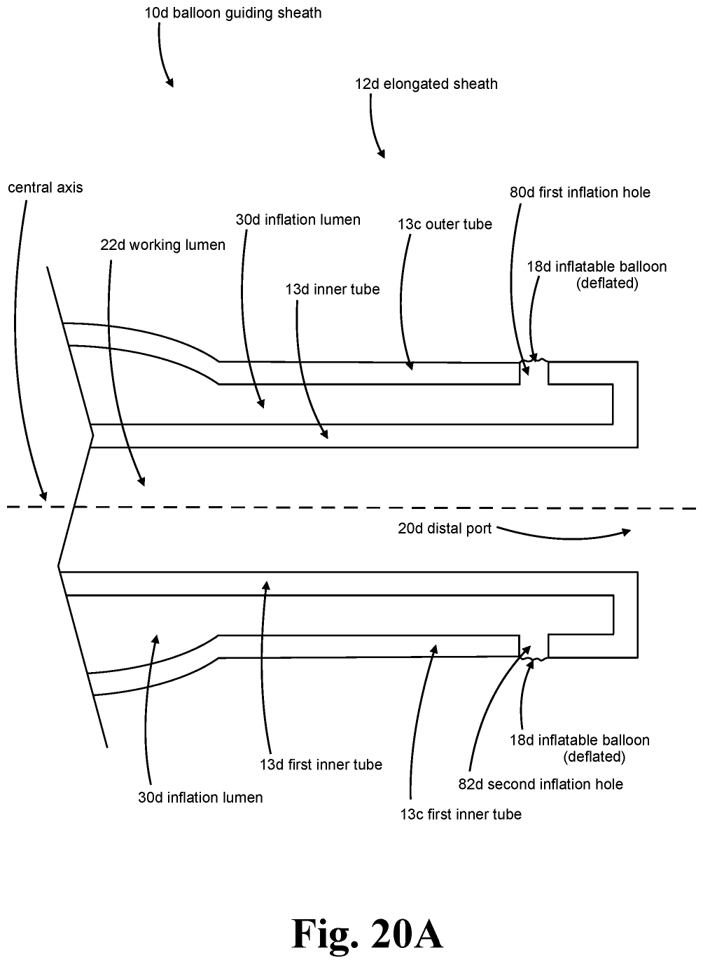

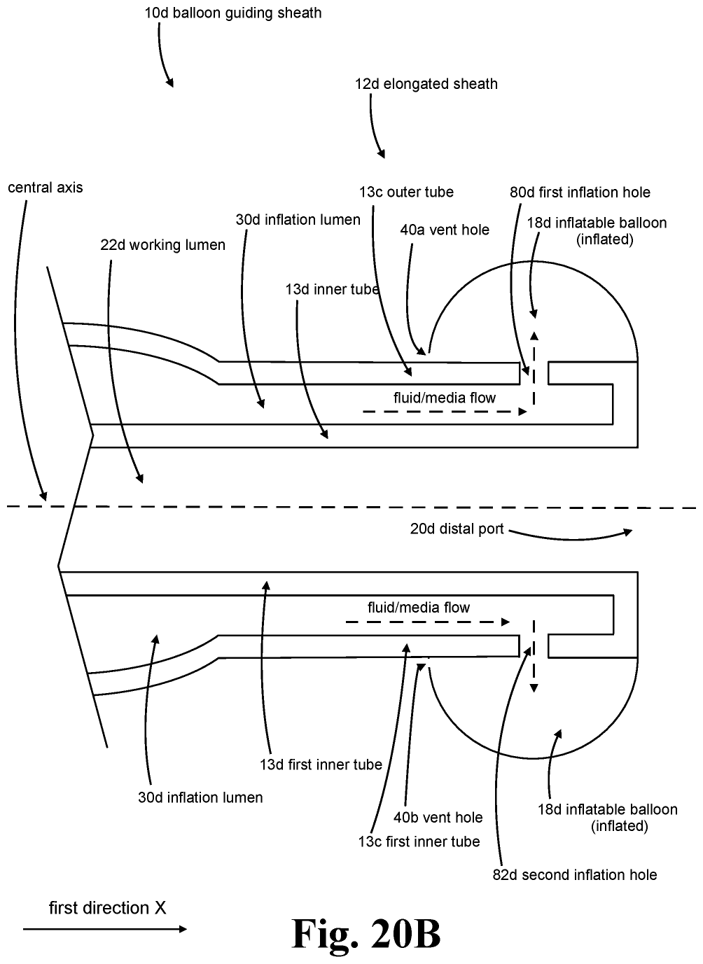

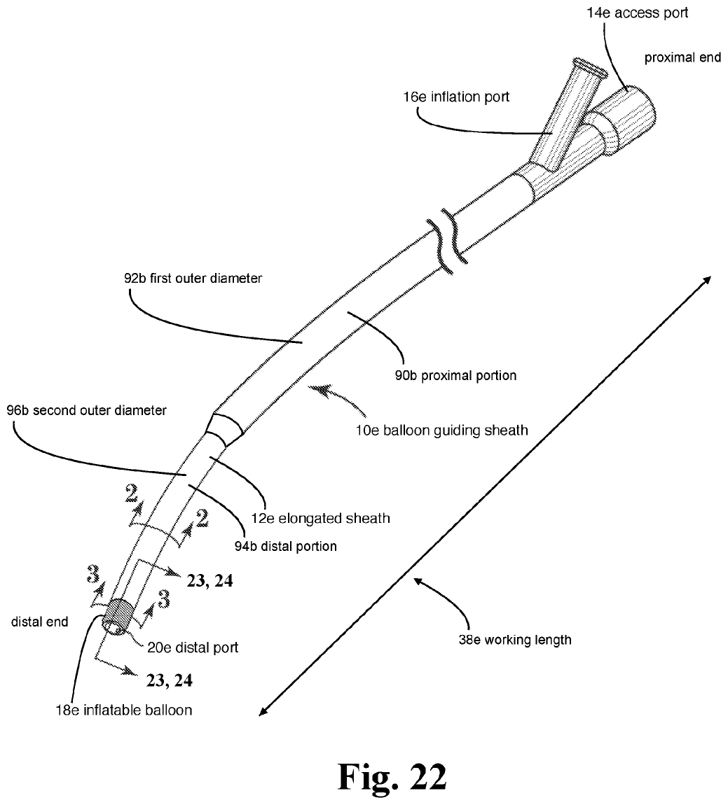

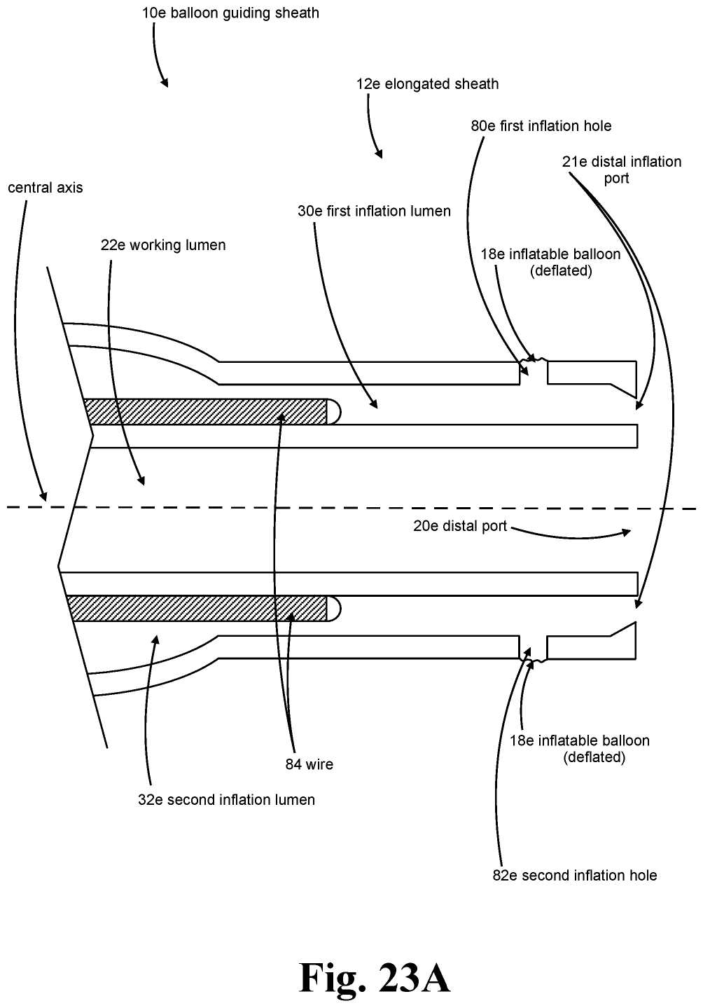

A balloon guiding sheath 10d in accordance with embodiments of the invention can be described generally with reference to FIGS. 19-21. As shown in FIG. 19, a balloon guiding sheath 10d may comprise an elongated sheath 12d having a proximal end, a distal end, a proximal portion 90a defining a first outer diameter 92a, and a distal portion 94a defining a second outer diameter 96a. With reference to FIGS. 20A and 20B, the elongated sheath 12d may include an inner tube 13c and an outer tube 13d that surrounds the inner tube 13c. It should be appreciated that many embodiments may also be implemented with other tube arrangements, as will be discussed later regarding FIGS. 22-24. Other embodiments may include two, three, or four or more tubes, also referred to as layers.

Similar to other embodiments described throughout this disclosure, the components of the balloon guiding sheath 10d may be formed from a polymer (e.g. polytetrafluoroethylene, nylon, and the like). In some embodiments, the components may comprise Pellethane 65D or higher. Generally, the material selection may be focused on enhancing pushability in the balloon guiding sheath 10d as opposed to flexibility. However, it should be appreciated that in some embodiments the material selection may be focused on either or both pushability and/or flexibility. Generally, in many embodiments, the elongated sheath 12d is arranged and configured to have sufficient stiffness and tip flexibility to enable insertion of the working length 38d of the elongated sheath 12d into a patient's vasculature through an arteriotomy in the patient's femoral artery to position the distal port 20 at a target site in at least one of a petrous portion 54 of a patient's internal carotid artery 50, a cavernous portion 56 of a patient's internal carotid artery 50, and a cerebral portion 58 of a patient's internal carotid artery 50.

As shown in FIG. 19, the guiding sheath 10d may include an access port 14d located on the proximal end and an inflation port 16d also located on the proximal end. The guiding sheath 10d may include an inflatable balloon 18d coupled to the distal end and a distal port 20d also located on the distal end. As shown in FIGS. 20a and 20b, the guiding sheath 10d may include a working lumen 22d extending through the elongated sheath 12 between the access port 14d and the distal port 20d.

Furthermore, the guiding sheath 10d may include an inflation lumen 30d that extends between the inflation port 16d and the balloon 18d. In the embodiment disclosed with respect to FIGS. 19-21, the inflation lumen 30d is located between the inner tube 13c and the outer tube 13d. As illustrated, the inflation lumen 30d is not in fluid communication with the working lumen 22d.

With reference back to FIG. 2, the guiding sheath 10d may include a reinforcement layer 31 located between the inner tube 13c and the outer tube 13d. The reinforcement layer 31 may be arranged and configured to enable flow of at least one of fluid and media through the inflation lumen 30d to thereby inflate the balloon 18d. The reinforcement layer 31 may be comprised of coiled and/or braided strands of material (e.g. stainless steel or polymer wire). It should be appreciated that any of the components described with respect to different embodiments may thereby be implemented with respect to any embodiment described throughout this disclosure.

Now with reference to FIGS. 20A and 20B, a cross-sectional side view of section 20-20 is shown. As shown, the balloon 18d may move between a deflated state (FIG. 20A) and an inflated state (FIG. 20B). The inflation and deflation may occur in response to fluid and/or media traveling through the inflation lumen 30d, through a respective inflation hole(s) 80d, 82d (e.g. first inflation hole 80d and second inflation hole 82d), and into the balloon 18d. In such embodiments, the inflation lumen 30d is not in fluid communication with working lumen 22d. In this regard, the balloon 18d may inflate and deflate irrespective of any interaction with the working lumen 22d. In doing so, the clinician may thereby occlude blood flow through the patient's artery while moving devices and tools through the working lumen 22d without interfering with balloon 18d inflation and deflation.

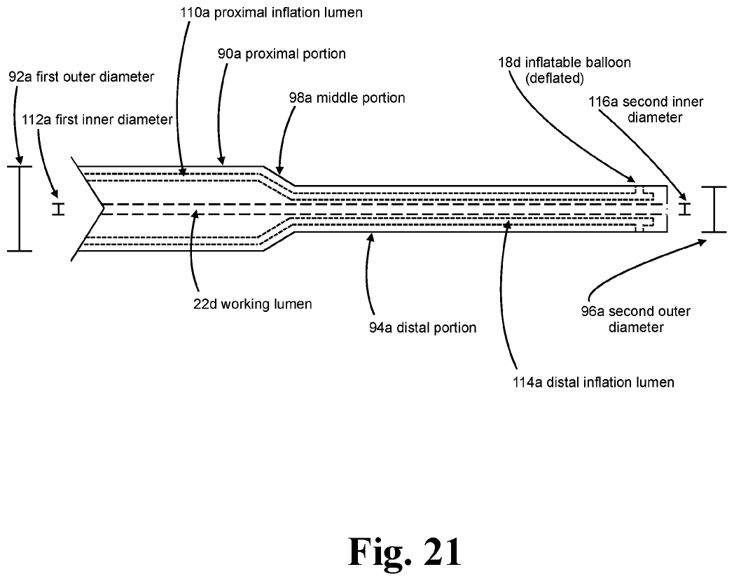

In many embodiments, the elongated sheath 12d is sized and configured to enable direct insertion into a patient's vasculature through an arteriotomy in the patient's femoral artery to position the balloon 18d at a target site. As shown in FIG. 21, which illustrates a cross-sectional side view of section 21-21, the first outer diameter 92a may be greater than the second outer diameter 96a. As such, the elongated sheath 12d may not have a generally constant outer diameter along its working length 38d.

With regard to specific dimensions, in some embodiments, the first outer diameter 92a is approximately equal to 0.123 inches. Even still, in some embodiments, the first outer diameter 92a is approximately equal to 0.137 inches. The second outer diameter 96a may be approximately equal to 0.104 inches such that the elongated sheath 12d fits through an 8 French (Fr) opening. In some embodiments, the second outer diameter 96a is approximately equal to 0.124 inches such that the elongated sheath 12d fits through a 10 French (Fr) opening. Generally, it should be appreciated that the first outer diameter 92a, second outer diameter 96a, and any other dimension recited in this disclosure may be equal to any value based on medical application and patient anatomy.

With continued reference to FIG. 21, the inflation lumen 30d may include a proximal inflation lumen 110a, indicated by broken lines, which extends from the inflation port 16d to a middle portion 98a located between the proximal portion 90a and the distal portion 94a. Additionally, the inflation lumen 30d may include a distal inflation lumen 114a, represented in broken lines, which extends from the middle portion 98a to the balloon 18d. In this regard, the distal inflation lumen 114a may be located between the inner tube 13c and the outer tube 13d.

Again, in regards to various commercial embodiments, the proximal and distal portions 90a, 94a may define a variety of different dimensions arranged and configured to meet the needs of specific applications and patient anatomies. For example, the working lumen 22d may define an inner diameter having a substantially constant diameter from the distal end to the proximal end. Specifically, in some embodiments, the inner diameter is approximately equal to 0.088 inches.

However, in many embodiments, the working lumen 22d does not define a constant inner diameter from the distal end to the proximal end. In some embodiments, the proximal portion 90a of the working lumen 22d defines a first inner diameter 112a approximately equal to 0.090 inches, while the distal portion 94a of the working lumen 22d defines a second inner diameter 116a greater than or equal to 0.086 inches and less than or equal to 0.090 inches. Even still, in some embodiments, the first inner diameter 112a is greater than or equal to 0.101 inches and less than or equal to 0.113 inches, and the second inner diameter 116a is greater than or equal to 0.101 inches and less than or equal to 0.113 inches. With even more specificity, in some embodiments, the first inner diameter 112a is approximately equal to 0.113 inches and the second inner diameter 116a is approximately equal to 0.101 inches.

The elongated sheath 12d may define a variety of working lengths sized and configured to accommodate treatment locations in different target sites. In some embodiments, the working length 38d is long enough to enable the distal end to reach at least a cervical portion 52 of a patient's internal carotid artery 50 from the femoral artery. The working length 38d may be long enough to enable the distal end to reach at least a petrous portion 54 of the patient's internal carotid artery 50 from the femoral artery. Even still, the working length 38d may be long enough to enable the distal end to reach at least a cavernous portion 56 of the patient's internal carotid artery 50 from the femoral artery. In some embodiments, the working length 38d is approximately equal to 95 centimeters. However, it should be appreciated that the working length 38d may be any length less than or greater than 95 centimeters. In some embodiments, the proximal portion 90a of the working lumen 22d equals approximately 85 centimeters in length, while the distal portion 94a of the working lumen 22d equals approximately 10 centimeters in length. However, the lengths of the proximal portion 90a and the distal portion 94a may define any length such that they equal the overall working length 38d.

BOSS Max and BOSS Slender Tip-Occluding Embodiments

Yet another balloon guiding sheath 10e in accordance with embodiments of the invention is now described with reference to FIGS. 22-24. As shown in FIG. 22, the guiding sheath 10e includes an elongated sheath 12e having a proximal end and a distal end. The guiding sheath 10e may include an access port 14e located on the proximal end and a distal port 20e located on the distal end. As well, the guiding sheath 10e includes a working lumen 22e extending through the elongated sheath 12e between the access port 14e and the distal port 20e. Similar to other embodiments disclosed, the guiding sheath 10e includes an inflation port 16e located on the proximal end and an inflation lumen 30e extending through the elongated sheath 12e between the inflation port 16e and the balloon 18e.

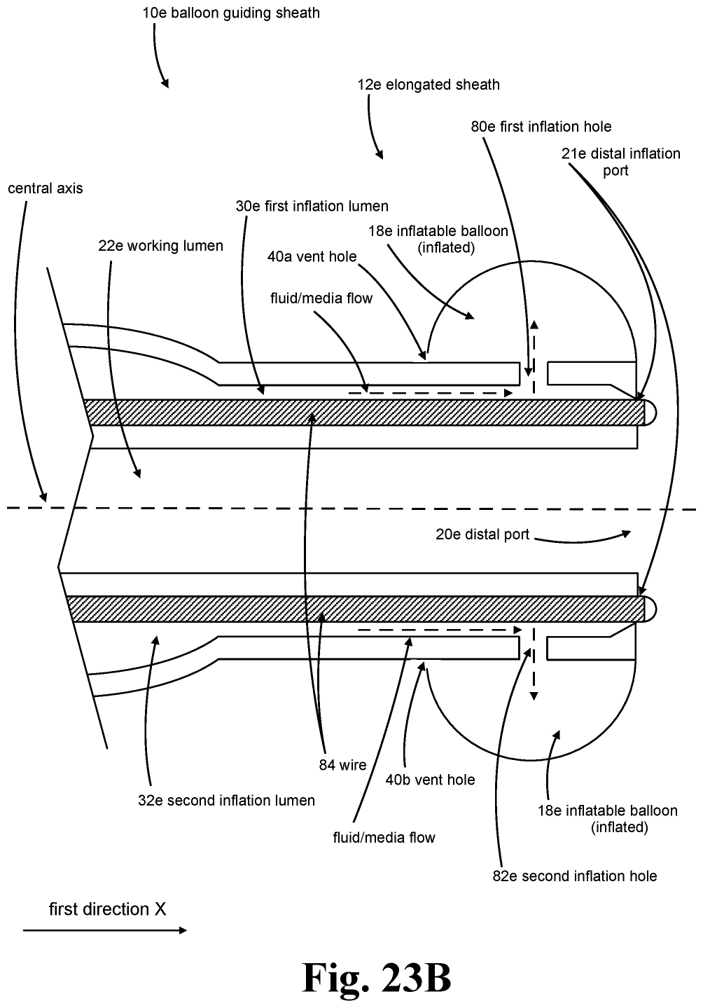

With respect to FIGS. 23A and 23B, which illustrate a cross-sectional side view of section 23-23, the inflation lumen 30e may include a distal inflation port 21e extending through an endwall of the elongated sheath 12e. The inflation lumen 30e may not be in fluid communication with the working lumen 22e between the access port 14e and the distal port 20e. The guiding sheath 10e may also include one or more inflation hole(s) 80e, 82e extending from the inflation lumen 30e through a sidewall of the elongated sheath 12e and into the balloon(s) 18e.

Similar to the embodiment described with respect to FIGS. 12A and 12B, when a wire 84 is inserted into the inflation lumen 30e and out through the distal inflation port 21e, the distal inflation port 21e may thereby create a seal against the guide wire 84. Once the seal is created, the inflation lumen 30e may enable flow of at least one of fluid and media through the inflation lumen 30e into the one or more inflation hole(s) 80e, 82e and into the balloon 18e to thereby inflate the balloon 18e, as shown in FIG. 23B.

In some embodiments, the elongated sheath 12e includes more than one inflation lumen. For example, the balloon guiding sheath 10e may thereby include a second inflation lumen 32e extending through the elongated sheath 12e between the inflation port 16e and the balloon 18e. The second inflation lumen 32e may include a second distal inflation port 21e extending through the endwall of the elongated sheath 12e. In such embodiments, the second inflation lumen 32e is not in fluid communication with the working lumen 22e between the access port 14e and the distal port 20e. The guiding sheath 10e may thereby include a second inflation hole 82e extending from the second inflation lumen 32e through the sidewall of the elongated sheath 12e. Similar to the inflation technique described above, when a second wire 84 is inserted into the second inflation lumen 32e and out through the second distal inflation port 21e, the second distal inflation port 21e may thereby create a seal against the second wire 84. Once this occurs, the flow of fluid and/or media through the second inflation lumen 32e into the second inflation hole 82e and into the balloon 18e thereby inflates the balloon 18e.

Regarding specific orientations of the working lumen 22e with respect to the inflation lumen 30e, the elongated sheath 12e may define a central axis extending from the proximal end to the distal end. As shown in FIGS. 23A and 23B, at least a portion of the working lumen 22e may overlap the central axis of the elongated sheath 12e, while the inflation lumen 30e does not overlap the central axis of the elongated sheath 12e. As such, the inflation lumen 30e may be radially spaced from the working lumen 22e.

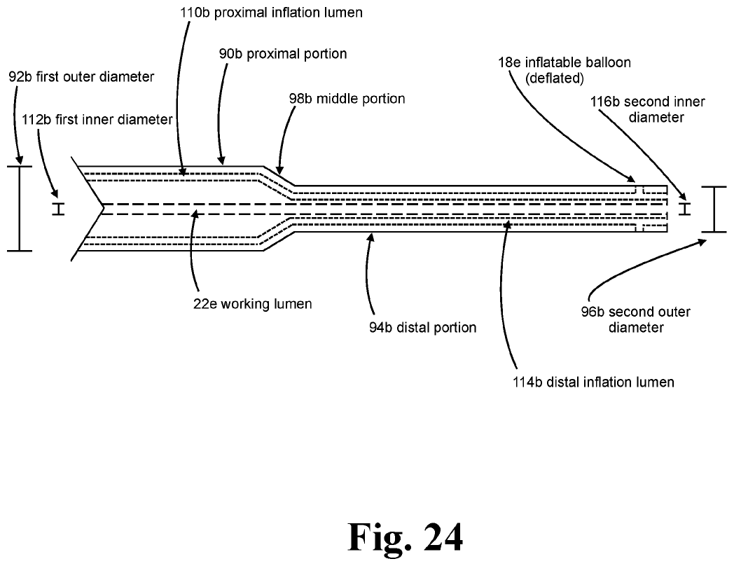

The elongated sheath 12e may be sized and configured to enable direct insertion into a patient's vasculature through an arteriotomy in the patient's femoral artery to position the balloon 18e at a target site. As shown in FIG. 24, which illustrates a cross-sectional side view of section 24-24, the elongated sheath 12e may have a proximal portion 90b defining a first outer diameter 92b, and a distal portion 94b defining a second outer diameter 96b. In some embodiments, the first outer diameter 92b may be greater than the second outer diameter 96b. As such, the elongated sheath 12e may not have a generally constant outer diameter, and instead have a varying outer diameter along its working length 38e.

With continued reference to FIG. 24, the inflation lumen 30e may include a proximal inflation lumen 110b, represented by the broken lines, which extends from the inflation port 16e to a middle portion 98b located between the proximal portion 90b and the distal portion 94b. Additionally, the inflation lumen 30e may include a distal inflation lumen 114b, represented by the broken lines, which extends from the middle portion 98b to the balloon 18e. In some embodiments, the proximal portion 90b of the working lumen 22e defines a first inner diameter 112b, while the distal portion 94b of the working lumen 22e defines a second inner diameter 116b.

Generally, embodiments disclosed with respect to FIGS. 22-24 may define any of the dimensions as previously defined throughout this disclosure. Generally, the guiding sheath 10e may be sized and configured such that the elongated sheath defines a working length long enough to enable the distal end to reach at least a cervical portion 52 of a patient's internal carotid artery 50 from the carotid artery. As well, the working length may be long enough to enable the distal end to reach a cavernous portion 56 of the patient's internal carotid artery 50 from the femoral artery.

BOSS Max and BOSS Slender Method Embodiments

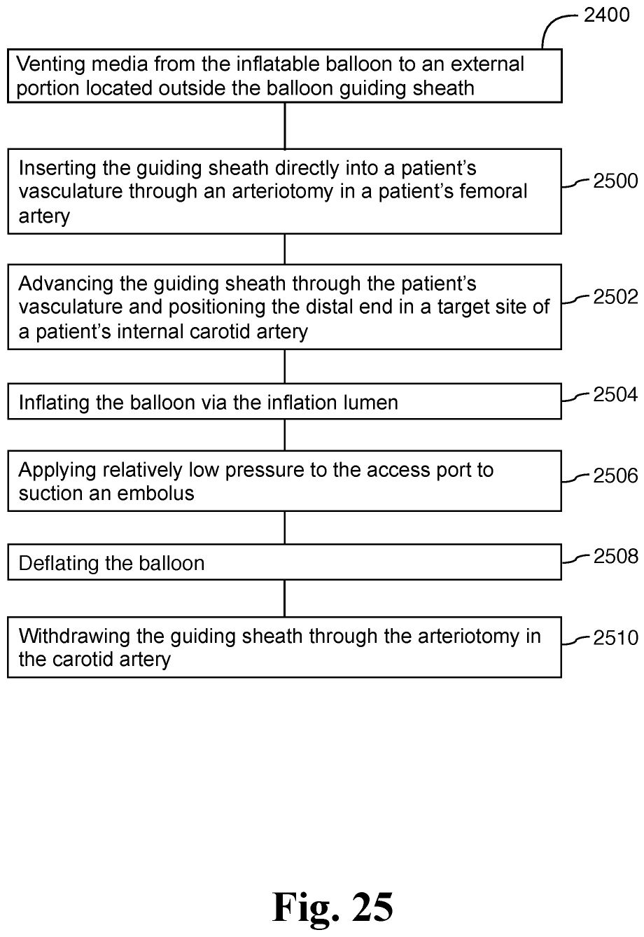

With reference to FIGS. 25-27, the disclosure also includes methods for using the balloon guiding sheaths 10d, 10e as described above. Some methods may be implemented with either guiding sheath 10d or 10e. However, some methods may only be implemented with embodiment 10e. Each circumstance is described in below.

As shown in FIG. 25, methods may include inserting the guiding sheath 10d, 10e directly into a patient's vasculature through an arteriotomy in a patient's femoral artery (at step 2500), advancing the guiding sheath 10d, 10e through the patient's vasculature, and positioning the distal end in a target site of a patient's internal carotid artery (at step 2502). Methods may also include inflating the balloon 18d, 18e via the inflation lumen 30d, 30e (at step 2504). It should be appreciated that the target site may be a cervical portion 52 of the internal carotid artery 50, a petrous portion 54 of the internal carotid artery 50, and/or a cavernous portion 56 of the internal carotid artery 50.

As further shown, many methods include applying relatively low pressure to the access port 14d, 14e to suction an embolus (at step 2506). Once this is complete, methods may include deflating the balloon (at step 2508) and thereby withdrawing the guiding sheath 10d, 10e through the arteriotomy in the carotid artery (at step 2510).

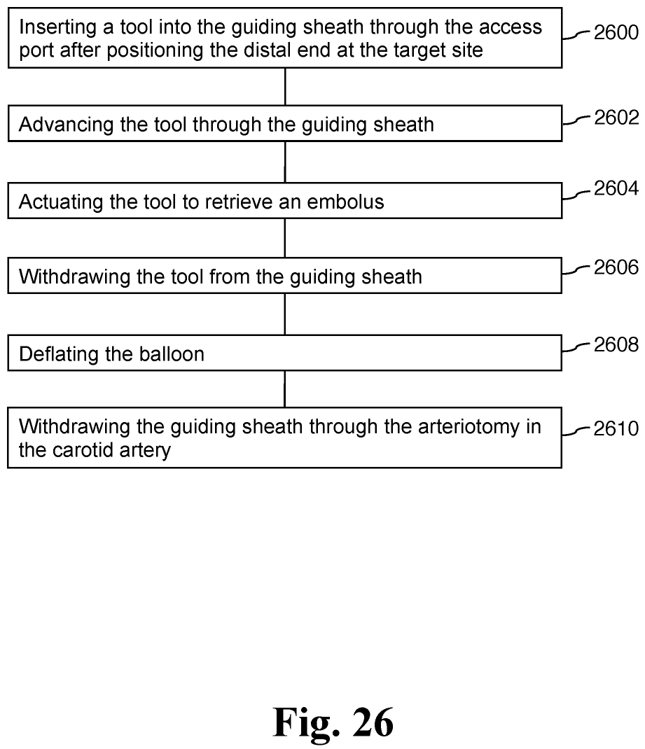

However, clinicians may need to perform other treatments to the target site, which may require the use of additional equipment. Accordingly, methods may include the following intervening steps that may occur after step 2504. As shown in FIG. 26, such intervening steps may include inserting a tool into the guiding sheath 10d, 10e through the access port 14d, 14e after positioning the distal end at the target site (at step 2600), and advancing the tool through the guiding sheath 10d, 10e (at step 2602). Methods may thereby include actuating the tool to retrieve the embolus (at step 2604) and withdrawing the tool from the guiding sheath 10d, 10e (at step 2606). Once complete, methods may include deflating the balloon 18d, 18e (at step 2608) and withdrawing the guiding sheath 10d, 10e through the arteriotomy in the carotid artery (at step 2610).

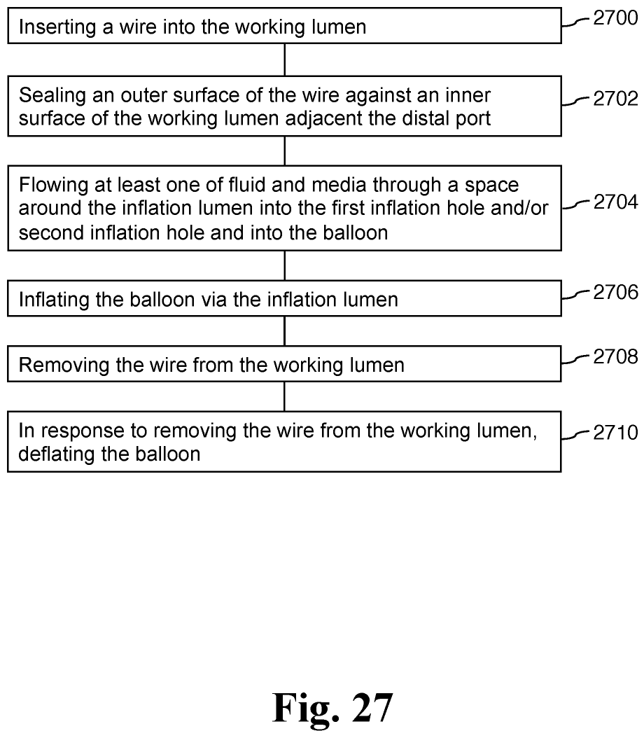

As shown in FIG. 27, the method may also include specific method steps to be performed with balloon guiding sheath 10e. In accordance, inflating the balloon 18e via the inflation lumen 30e occurs in response to inserting a guide wire 84 into the working lumen 22e (at step 2700) and sealing an outer surface of the guide wire 84 against an inner surface of the working lumen 22e adjacent the distal port 20e (at step 2702). Methods may also include flowing at least one media through a space within the inflation lumen 30e between the wire 84 and an inner surface of the inflation lumen 30 and into at least one inflation hole 80e, 82e and into the balloon 18e (at step 2704), thereby inflating the balloon 18e (at step 2706).

In some embodiments, methods may also include removing the wire 84 from the working lumen 22e (at step 2708) and in response to removing the wire 84 from the working lumen 22e, deflating the balloon 18e (at step 2710).

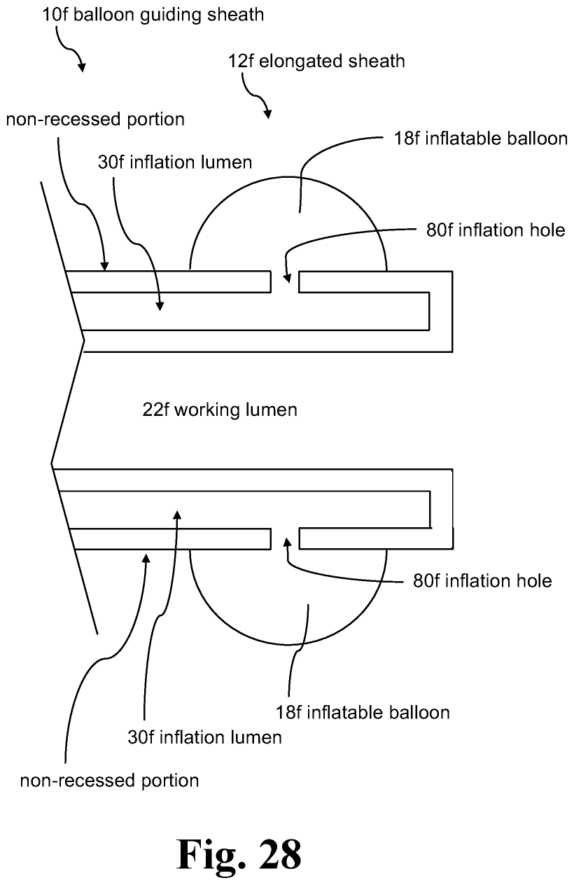

FIG. 28 illustrates yet another embodiment of a balloon guiding sheath 10f and elongated sheath 12f. As shown, the inflatable balloon 18f (shown in the inflated state) is located in a non-recessed portion of the outer surface of the elongated sheath 12f. In such embodiments, the outer surface, or outer profile, of the elongated sheath 12f may define a zero profile whereby the inflatable balloon 18f is flush bonded to the outer surface of the elongated sheath 12f. This embodiment is unique with respect to the prior art (U.S. Pat. Nos. 6,638,245 and 6,702,782 assigned to Concentric Medical, Inc., see FIGS. 4a and 4b) because the outer surface of the elongated sheath 12f, as shown in FIG. 28, is able to achieve a generally constant outer diameter without having to recess the portion of the elongated sheath located beneath the inflatable balloon 18f.

Interpretation

As presented in this disclosure, the term “substantially” shall mean “for the most part.” As such, if we say that a first object is substantially horizontally aligned with a second object this means that at least half a surface area of the first object overlaps with at least half a surface area of the second object. Within a numerical context “substantially” shall mean±0.001 inches. For example, to say that an inner diameter of a working lumen is substantially equal to 0.091 inches, means that the inner diameter is 0.091±0.001 inches.

As presented in this disclosure, the term “generally” shall be interpreted numerically as ±0.003 inches. For example, to say that an elongated sheath defines a generally constant outer diameter shall mean that the outer diameter is generally constant but may vary by ±0.003 inches. For example, if the outer diameter is 0.123 inches and generally constant shall mean that the outer diameter may vary between 0.126 inches and 0.120 inches.

Furthermore, as presented in this disclosure, the term “about” shall mean approximately or roughly. Within a numerical context “about” shall mean±0.5 centimeters. For example, to say that the length of a guiding sheath is about 90 centimeters, means that the guiding sheath is 90±0.5 centimeters.

None of the steps described herein is essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

The section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1 and embodiments described in other sections may apply to and be combined with embodiments described within the “Topic 1” section.

The various features and processes described above may be used independently of one another, or may be combined in various ways. All possible combinations and subcombinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, in parallel, or in some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments include A, B, and C. The term “and/or” is used to avoid unnecessary redundancy.

Although the embodiments herein are described with various specific embodiments, it will be obvious for a person skilled in the art to practice the invention with modifications. However, all such modifications are deemed to be within the scope of the claims. While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

Furthermore, the foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Implementation

The aforementioned embodiments can be implemented, for example, using a machine-readable medium or article which is able to store an instruction or a set of instructions that, if executed by a machine, can cause the machine to perform a method and/or operations described herein. Such machine can include, for example, any suitable processing platform, computing platform, computing device, processing device, electronic device, electronic system, computing system, processing system, computer, processor, or the like, and is able to be implemented using any suitable combination of hardware and/or software.

What is claimed is:

1. A balloon guiding sheath, comprising:
an elongated sheath having a proximal end, a distal end opposite the proximal end, an inner tube extending between the proximal end and the distal end, an outer tube surrounding the inner tube and extending between the proximal end and the distal end, an access port located adjacent the proximal end, a distal port located adjacent the distal end, and a working lumen extending through the elongated sheath between the access port and the distal port; and
an inflatable balloon located on an outer surface of the elongated sheath adjacent the distal end, the inflatable balloon being fluidly coupled to an inflation lumen extending between the inflatable balloon and an inflation port located adjacent the proximal end,
wherein the elongated sheath is sized and configured to enable direct insertion into a patient's vasculature through an arteriotomy in at least one of the patient's carotid artery and vertebral artery to position the balloon at a target site.

2. The balloon guiding sheath of claim 1, further comprising a reinforcement layer located between the inner tube and the outer tube, the reinforcement layer arranged and configured to enable flow of at least one of fluid and media through the inflation lumen.

3. The balloon guiding sheath of claim 1, wherein the elongated sheath defines a working length comprising a proximal portion, a distal tip, and a distal portion located between the proximal portion and the distal tip.

4. The balloon guiding sheath of claim 3, wherein the elongated sheath defines a generally constant outer diameter from the proximal portion to the distal portion.

5. The balloon guiding sheath of claim 4, wherein the generally constant outer diameter is equal to 0.123 inches.

6. The balloon guiding sheath of claim 4, wherein the generally constant outer diameter is equal to 0.110 inches.

7. The balloon guiding sheath of claim 4, wherein the generally constant outer diameter is equal to 0.102 inches.

8. The balloon guiding sheath of claim 3, wherein the elongated sheath defines a generally constant inner diameter from the proximal portion to the distal portion.

9. The balloon guiding sheath of claim 8, wherein the generally constant inner diameter is equal to 0.103 inches.

10. The balloon guiding sheath of claim 8, wherein the generally constant inner diameter is equal to 0.088 inches.

11. The balloon guiding sheath of claim 8, wherein the generally constant inner diameter is equal to 0.087 inches.

12. The balloon guiding sheath of claim 3, wherein the elongated sheath defines an outer diameter that tapers downward from the proximal portion to the distal portion.

13. The balloon guiding sheath of claim 1, wherein the inflatable balloon is located in a non-recessed portion of the outer surface.

14. The balloon guiding sheath of claim 3, wherein the working length is about 95 centimeters.

15. The balloon guiding sheath of claim 1, wherein the elongated sheath defines a working length long enough to enable the distal port to reach at least a cervical portion of a patient's internal carotid artery from the carotid artery.

16. The balloon guiding sheath of claim 15, wherein the working length is long enough to enable the distal port to reach a cavernous portion of the patient's internal carotid artery from the carotid artery.

17. The balloon guiding sheath of claim 1, wherein the elongated sheath is arranged and configured to have sufficient stiffness and tip flexibility to enable insertion of the working length of the sheath into a patient's vasculature through an arteriotomy in the patient's carotid artery to position the distal port at a target site in at least one of a petrous portion of a patient's internal carotid artery, a cavernous portion of the patient's internal carotid artery, and a cerebral portion of the patient's internal carotid artery.
